(19) United States
(12) Patent Application Publication
(10) Pub. No.: US 2011/0129423 A1
(43) Pub. Date: Jun. 2, 2011

(54) ANTICANCER COMPOUNDS AND SCREENING METHOD

(76) Inventor: James M. Frincke, San Diego, CA (US)

(21) Appl. No.: 12/957,273

(22) Filed: Nov. 30, 2010

Related U.S. Application Data
(60) Provisional application No. 61/265,294, filed on Nov. 30, 2009.
(60) Provisional application No. 61/266,092, filed on Dec. 2, 2009.
(60) Provisional application No. 61/266,291, filed on Dec. 3, 2009.
(60) Provisional application No. 61/266,416, filed on Dec. 3, 2009.
(60) Provisional application No. 61/266,483, filed on Dec. 3, 2009.

(51) Int. Cl. A61K 49/00; A61P 35/00; A61P 43/00

(52) U.S. Cl. 424/9.2

(57) ABSTRACT

The invention provides a method to identify or characterize compounds for treating cancer wherein the compounds have a capacity to decrease systemic levels of one or more cholesterol metabolites in treated mammal(s). Such compounds include 17-(3-pyridyl)androst-5,16-diene-3β,7β-diol, 17-(2-morpholinyl)-7β-ethylandrost-5,16-diene-3β-ol, 17-(2-morpholinyl)-7β-ethylandrost-5,16-diene-3β-ol-16-acetate, 17-(4-oxazolyl)-7β-ethylandrost-5,16-diene-3β-ol-16-methyl ether and 17-(4-oxazolyl)-7β-ethylandrost-5,16-diene-3β-ol. Compositions and formulations comprising the compounds and one or more excipients are also provided.

### CROSS REFERENCE TO RELATED APPLICATIONS

This nonprovisional U.S. patent application claims priority from pending U.S. provisional application Ser. No. 61/265,294, filed Nov. 30, 2009, pending U.S. provisional application Ser. No. 61/266,092, filed Dec. 2, 2009, pending U.S. provisional application Ser. No. 61/266,291, filed Dec. 3, 2009, pending U.S. provisional application Ser. No. 61/266,416, filed Dec. 3, 2009, and pending U.S. provisional application Ser. No. 61/266,483, filed Dec. 3, 2009, all of which are incorporated herein by reference.

### FIELD OF THE INVENTION

The invention relates to methods to screen for and/or characterize compounds with activity as anticancer drugs that affect hormone signaling in vivo, including signaling by one or more steroid hormones.

### BACKGROUND

Anticancer drugs and treatments typically are accompanied by significant toxicity or unwanted side-effects that limit the usefulness of drug treatments. Methods to identify additional compounds that are useful in treating cancers are needed, particularly for common cancers and related conditions, e.g., lung cancer, colon cancer and neuroendocrine cancers such as prostate cancer and breast cancer.

### DESCRIPTION OF THE INVENTION

Summary of invention embodiments. In some embodiments, the invention provides a method to identify a compound comprising (a) administering a test compound to a mammal(s) for a sufficient period of time to obtain treated mammal(s); (b) measuring systemic levels of one or more cholesterol metabolites in the treated mammal(s); and (c) selecting the compound of step (b) that decreases the systemic levels of one or more cholesterol metabolites in the treated mammal(s), whereby a compound having a potential to treat a cancer, optionally a neuroendocrine disorder or tumor is identified, wherein the test compound of step (a) has the structure

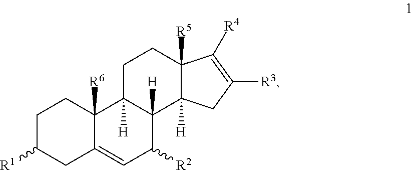

wherein, R¹ is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate), or an optionally substituted ether (including —O-optionally substituted C1-8 alkyl or —O-optionally substituted aryl, including —O-optionally substituted phenyl, optionally a C1-6 ether, including —OCH₃, —OC₂H₅, —OCH₂CH₂CH₃, —OCH₂CH₂OH, —OCH₂CH₂CH₂OH or —OCH(CH₃)₂); R² is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate), or an optionally substituted ether (including —O-optionally substituted C1-8 alkyl or —O-optionally substituted aryl, including —O-optionally substituted phenyl, optionally a C1-6 ether, including methoxy or ethoxy), or optionally substituted C1-8 alkyl (including —CH₃, —CF₃, —C₂H₅, —CH₂CH₂OH, —CH₂CH₂CH₃, —CH₂CH₂CH₂OH or —OCH(CH₃)₂) or R² may also be —H when (i) R³ is not —H, (ii) R⁵ is —C₂H₅ or —CH₂OH and/or (iii) R⁶ is —H, —C₂H₅ or —CH₂OH; R³ is —H, —OH, C₁₋₈ optionally substituted alkyl, optionally methyl, ethyl, n-propyl, i-propyl or 3-hydroxy-n-propyl, an optionally substituted ester (including —O—C(O)-optionally substituted C1-7 alkyl and —O—C(O)-optionally substituted aryl, including —O—C(O)-optionally substituted phenyl, optionally a C2-6 ester, including acetate or propionate, or benzoate), an optionally substituted ether (including —O-optionally substituted C1-8 alkyl or —O-optionally substituted aryl, including —O-optionally substituted phenyl, optionally a C1-6 ether (including —OCH₃, —OC₂H₅, —OCH₂CH₂CH₃, —OCH₂CH₂OH, —OCH₂CH₂CH₂OH or —OCH(CH₃)₂), or optionally substituted C1-8 alkyl (including —CH₃, —CF₃, —C₂H₅, —CH₂CH₂OH, —CH₂CH₂CH₃, —CH₂CH₂CH₂OH or —CH(CH₃)₂); R⁴ is —NH₂, —NHCH₃, —N(CH₃)₂, —NH—C(O)CH₃, —NHOH, an N-linked amino acid, C1-8 alkyl, optionally methyl, ethyl, n-propyl or i-propyl, a C-linked ring or an N-linked ring; R⁵ is —CH₃, —C₂H₅ or —CH₂OH; and R⁶ is —H, —CH₃, —C₂H₅ or —CH₂OH. Other compounds or compositions that can be used in the method are described elsewhere, e.g., in the claims. The method may optionally include treatment of the mammal(s) with vehicle (negative control) and/or other treatment control compounds, e.g., the 17α-hydroxylase/17,20 lyase inhibitor (CYP17A1), abiraterone or 17α-ethynylandrostane-3α,17β-diol.

The cholesterol metabolites include one or more of testosterone, dihydrotestosterone, 4-androstenedione, 5-androstenediol, 5α-androstane-3α,17β-diol, 5α-androstane-3β,17β-diol, estradiol, estrone, dehydroepiandrosterone (DHEA), pregnenolone, progesterone and cortisol, optionally wherein the cholesterol metabolites are one, two or more of (i) testosterone, dihydrotestosterone, 4-androstenedione and 5-androstenediol, (ii) estradiol, estrone and 4-androstenedione or (iii) pregnenolone, progesterone and cortisol. In preferred embodiments, the decreased cholesterol metabolite is not progesterone and/or cortisol.

Other embodiments include a drug product for treating a cancer, or a neuroendocrine disorder or tumor in a human comprising, (a) a drug in a dosage form, optionally wherein the dosage form is a formulation for oral, parenteral or topical administration; and (b) packaging for the drug together with a package insert or label that includes information about the drug's efficacy, toxicity or mechanism of action wherein such information was obtained at least in part from a method comprising (A) administering a test compound to a mammal(s) for a sufficient period of time to obtain treated mammal(s); (B) measuring systemic levels of one or more cholesterol metabolites in the mammal(s); and (C) selecting a candidate compound that decreases the systemic levels of one or more cholesterol metabolites in the treated mammal(s).

In these and related embodiments, the mammal(s) can be a feline but is preferably a rodent(s) or canine(s), optionally a mouse or rat. In some preferred embodiments, the mammal(s) will not contain tumors, e.g., prostate xenograft tumors in mice, to allow assessment of effects of the test compound on normal animals. In other embodiments, the mammal(s) will have a spontaneous or implanted tumor and these animals can be used to assess the activity of the test compound on the tumor or cancer in such an in vivo environment.

In preferred embodiments, the cancer or neuroendocrine disorder or tumor is prostate cancer, breast cancer or small cell lung cancer. In other embodiments, the neuroendocrine disorder or tumor is endometriosis or uterine fibroids.

### DETAILED DESCRIPTION

As used herein and unless otherwise stated or implied by context, terms that are used herein have the meanings defined below. Unless otherwise contraindicated or implied, e.g., by including mutually exclusive elements or options, in these definitions and anywhere the specification, claims or elsewhere herein, the terms “a” and “an” mean one or more and the term “or” means and/or, e.g., one or the other or both or all.

The phrase “C1-8 optionally substituted alkyl” means a linear or branched group or moiety containing 1, 2, 3, 4, 5, 6, 7 or 8 carbon atoms and one or more substituents, including —OH, halogen or ═O, that replace one or more hydrogen atoms, e.g., —CH₂CH₂CH₂OH. Preferably no more than one or two oxygen atoms are present. Preferred optionally substituted alkyl groups are C1-4 optionally substituted alkyl, which have 1, 2, 3 or 4 carbon atoms and 0 or 1 hydroxyl groups. Optionally substituted alkyl moieties are preferably saturated, but may contain a double bond(s) or triple bond(s), including alkyl moieties —CH₂CH═CH₂ or —CH₂CECH, C1-6 optionally substituted alkyl means moieties having 1, 2, 3, 4, 5 or 6 carbon atoms.

“Alkyl”, “alkyl group”, “alkyl moiety” and the like as used herein means a collection of linked carbon atoms and include linear, branched or cyclic carbon chains or any combination thereof. Alkyl moieties, as used herein, may further contain unsaturation, i.e., the alkyl group may comprise one, two, three or more independently selected double bonds or triple bonds. Unsaturated alkyl groups contain moieties as described for alkenyl and alkynyl moieties are described below. Preferred unsaturated alkyl groups contain one alkenyl moiety or one alkynyl moiety. The number of linked carbon atoms in an alkyl group or moiety can vary and typically is 1 to about 50, e.g., about 1-30 or about 1-20 linked carbon atoms, unless otherwise specified, e.g., C₁₋₈ alkyl or C1-C8 alkyl means an alkyl moiety containing 1, 2, 3, 4, 5, 6, 7 or 8 linked carbon atoms, C₁₋₇ alkyl or C1-C7 alkyl means an alkyl moiety containing 1, 2, 3, 4, 5, 6 or 7 linked carbon atoms and C₁₋₄ alkyl or C1-C4 alkyl means an alkyl moiety containing 1, 2, 3 or 4 linked carbon atoms. When an alkyl group is specified as a variable group, as for R¹, R², R³ or R⁴ variable group substituents described herein, a saturated carbon of the alkyl moiety is directly attached to the site occupied by the variable group, as in the C1-, C7-, C16- or C17-position of a steroid ring system, using the numbering convention for cholesterol, as described herein

When an alkyl group is specified as a variable group substituent, species may include methyl, ethyl, 1-propyl (n-propyl), 2-propyl (i-propyl, —CH(CH₃)₂), 1-butyl (n-butyl), 2-methyl-1-propyl (i-butyl, —CH₂CH(CH₃)₂), 2-butyl (s-butyl, —CH(CH₃)CH₂CH₃), 2-methyl-2-propyl (t-butyl, —C(CH₃)₃), 1-pentyl (n-pentyl), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl, 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂)_{,} 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), cyclopropyl (—CH<CH₂CH₂), cyclobutyl (—CH<CH₂CH₂CH₂), cyclopentyl, cyclohexyl, cycloheptyl, cyclooctyl, —(CH₂)ₙ—(CHCH₃)ₘ—(CH₂)ₒ—CH₃, —(CH₂)ₙ—(CHC₂H₅)ₘ—(CH₂)ₒ—CH₃ where n, m and o independently are 0, 1, 2, 3, 4, 5, 6, 7 or 8. Preferred alkyl moieties are C₁₋₈, C₁₋₇ or C₁₋₄ alkyl groups, including —CH₃ (methyl), —CH₂CH₃ (ethyl), —CH₂CH₂CH₃ (propyl), —CH(CH₃)₂ (isopropyl) and —CH₂CH₂CH₂CH₃ (butyl).

“Alkenyl”, “alkenyl group”, “alkenyl moiety” and the like as used herein means a collection of linked carbon atoms that contains one or more double bonds (e.g., —CH═CH— or ═CH— moieties), e.g., 1, 2, 3, 4, 5, 6 or more, preferably 1 or 2. An alkenyl group may further contain saturated carbons including linked normal, secondary, tertiary or cyclic carbon atoms that form linear, branched or cyclic carbon chains, which may also include the double bond moiety (e.g., —CH═CH-moiety) or any combination thereof or may contain other unsaturation including a triple bond moiety (i.e., —C≡C— moiety). Preferred alkenyl groups contain 0 or 1 additional alkenyl or 0-1 alkynyl moieties. The number of linked carbon atoms in an alkenyl group or moiety can vary and typically is 2 to about 50, e.g., about 2-30 or about 2-20, unless otherwise specified, e.g., C₂₋₈ alkenyl or C2-8 alkenyl means an alkenyl moiety containing 2, 3, 4, 5, 6, 7 or 8 carbon atoms and C₂₋₆ alkenyl or C2-6 alkenyl means an alkenyl moiety containing 2, 3, 4, 5 or 6 carbon atom. When an alkenyl group is specified as a variable group, as for R¹, R², R³ or R⁴ variable group substituents described herein, a unsaturated carbon of the alkenyl moiety is directly attached to the site occupied by the variable group, as in the C1-, C7-, C16- or C17-position of a steroid ring system, using the numbering convention for cholesterol, as described herein. When an alkenyl group is specified as a variable group substituent, species may include methylene (═CH₂), methylmethylene (═CH—CH₃), ethylmethylene (═CH—CH₂—CH₃), ═CH—CH₂—CH₂—CH₃, vinyl (—CH═CH₂), —(CCH₃═CH)—(CH₂)ₘ—CH₃, —(CH═CCH₃)—(CH₂)ₘ—CH₃ and —(CH═CH)—(CH₂)ₘ—CH₃, where m is 0, 1, 2, 3, 4, 5, 6, 7 or 8, preferably 0-3. Preferred alkenyl moieties are C₂₋₈, C₂₋₈ or C₂₋₄ alkenyl groups, including —CH═CH2 (vinyl), —CH═CH₂CH₃ ethenyl, —CH═HCH₂CH₃ (propenyl) and —CH═CH₂CH₂CH₃ (butenyl).

“Alkynyl”, “alkynyl group”, “alkynyl moiety” and the like as used herein means a collection of linked carbon atoms that contains one or more triple bonds (e.g., —C≡C— moiety), e.g., 1, 2, 3, 4, 5, 6 or more, preferably 1 or 2. An alkenyl group may further contain saturated carbons including linked normal, secondary, tertiary or cyclic carbon atoms that form linear, branched or cyclic carbon chains, which may also include the triple bond moiety, or any combination thereof, or may contain other unsaturation, including a double bond moiety (e.g., —CH═CH— moiety). Preferred alkynyl groups contain 0 or 1 additional alkynyl or 0-1 alkenyl moieties.

The number of linked carbon atoms in an alkenyl group or moiety can vary and typically is 2 to about 50, e.g., about 2-30 or about 2-20, unless otherwise specified, e.g., C₂₋₈ alkynyl or C2-8 alkynyl means an alkynyl moiety containing 2, 3, 4, 5, 6, 7 or 8 carbon atoms and C₂₋₆ alkynyl or C2-6 alkynyl means an alkynyl moiety containing 2, 3, 4, 5 or 6 carbon atoms. When an alkynyl group is specified as a variable group, as for R¹, R², R³ or R⁴ variable group substituents described herein, a unsaturated carbon of the alkynyl moiety is directly attached to the site occupied by the variable group, as in the C1-, C7-, C16- or C17-position of a steroid ring system, using the numbering convention for cholesterol, as described herein. When an alkynyl group is specified, groups and species may include —C≡CH, —C≡CCH₃, —C≡CCH₂CH₃, —C≡CC₃H₇, —C≡CCH₂C₃H₇, —(C≡C)—(CH₂)ₘ—CH₃, —(C≡C)₀₋₁—(CH₂)ₘ—CH₂C≡CH and —(C≡C)₀₋₁—(CH₂)ₙ—CH₂C═C(CH₂)ₘH, where m independently are 0, 1, 2, 3, 4, 5, 6, 7 or 8.

“Aryl”, “aryl group”, “aryl moiety” and the like means an aromatic ring with no ring heteroatoms and includes, phenyl, naphthyl or a carbocyclic ring system containing 2n+2 μl electrons where n is 0 or a positive integer. In some embodiments, the alkylaryl moiety is linked to a variable position of a steroid nucleus, replacing variable group substituents that include R¹, R², R³ or R⁴, i.e., alkyl-aryl-steroid, preferably R⁴.

“Arylalkyl” means a moiety where an alkyl group is bonded to an aryl group, i.e., -alkyl-aryl, where alkyl and aryl groups are as described above, e.g., —CH₂—C₆H₅ or —CH₂CH(CH₃)—C₆H₅. In some embodiments, the arylalkyl moiety is linked to a variable position of a steroid nucleus, replacing variable group substituents that include R¹, R², R³ or R⁴, i.e., aryl-alkyl-steroid

“Alkylaryl” means a moiety where an aryl group is bonded to an alkyl group, i.e., -aryl-alkyl, where aryl and alkyl groups are as described above, e.g., —C₆H₄—CH₃ or —C₆H₄—CH₂CH(CH₃). In some embodiments, the alkylaryl moiety is linked to a variable position of a steroid nucleus, replacing variable group substituents that include R¹, R², R³ or R⁴, i.e., alkyl-aryl-steroid, preferably R⁴.

“Heterocycle” “heterocycle group”, “heterocycle moiety” and the like includes by way of example and not limitation the heterocycles described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 1960, 82:5566, which are incorporated by reference herein. A heterocycle group or substituent is typically bonded to an organic moiety through a ring carbon atom or a ring nitrogen atom of the heterocycle. Heterocycle groups or substituents include aromatic (i.e., heteroaryl) and non-aromatic heterocycles. A heterocycle substituent attached to an organic moiety, including a steroid ring system, through a carbon of the heterocyclic ring is referred to as a C-linked heterocycle or a C-heterocycle (C-linked ring) and a heterocycle bonded through a nitrogen atom of the heterocyclic ring is referred to as an N-linked heterocycle or an N-heterocycle (N-linked ring). Preferred heterocycles are morpholine, piperidine, pyrazine, pyridine, pyrimidine, pyrrolidine, piperazine, imidazole, imidazolidin-2-one, dihydroimidazole, pyrrole, pyrazole, pyrazolone, thiazole, thiophene, pyran, oxolane and furan. For certain preferred heterocycle substituents, a C-heterocycle or an N-heterocycle is preferably bonded to the 17-position the steroid ring system (i.e., replaces R⁴ variable substituent).

“Heteroaryl”, “heteroaryl group”, “heteroaryl moiety” and the like means a heterocycle comprised of an aromatic ring where the aromatic ring contains 1, 2, 3 or more heteroatoms that participate in aromaticity of the ring, usually oxygen (—O—), nitrogen (—NX-), where X is —H, a protecting group, C₁₋₆ optionally substituted alkyl, an aryl or a heterocycle group, or sulfur (—S—), usually —H. Examples are as described for heterocycle. In some embodiments, the heteroaryl moiety is linked to a variable position of a steroid nucleus, replacing variable group substituents that include R¹, R², R³ or R⁴, i.e., heteroaryl-steroid, preferably R⁴.

“Substituted alkyl”, “substituted alkenyl”, “substituted alkynyl”, substituted alkylaryl”, “substituted arylalkyl”, “substituted heterocycle”, “substituted aryl”, substituted heteroaryl and the like mean an alkyl, alkenyl, alkynyl, alkylaryl, arylalkyl heterocycle, aryl, heteroaryl or other group or moiety as defined or disclosed herein that has a substituent(s) that replaces a hydrogen atom(s). Substituted heterocycles may thus have a substituent bonded to a ring carbon of the heterocycle or a ring heteroatom. Substituents for any of these moieties include 1, 2, 3, 4, 5, 6, 7, 8, 9, or 10 or more, preferable 1 or 2 independently selected heteroatoms, functional groups or other moieties described herein including nitrogen, oxygen, sulfur, phosphorous or silicon containing substituents, halogen, including —F, —Cl, Br or —I, or acyl, amine, amide, ester, carbamate, carbonate, alkoxy, aryl, heterocycle or heteroaryl containing substituents.

“Protecting group” means a moiety that prevents or reduces the atom or functional group to which it is linked from participating in unwanted reactions. For example, for —OR^{PR}, R^{PR} may be hydrogen or a protecting group for the oxygen atom found in a hydroxyl, while for —C(O)—OR^{PR}, R^{PR} may be hydrogen or a carboxyl protecting group, for —SR^{PR}, R^{PR} may be hydrogen or a protecting group for sulfur in thiols for instance, and for —NHR^{PR} or —N(R^{PR})₂-, R^{PR} may be hydrogen or a nitrogen atom protecting group for primary or secondary amines. Hydroxyl, amine, ketones and other reactive groups as found in variable group substituents for R¹, R², R³ or R⁴ described herein may require protection against reactions taking place elsewhere in the molecule. The protecting groups for oxygen, sulfur or nitrogen atoms are usually used to prevent unwanted reactions with electrophilic compounds, such as acylating agents used, e.g., in steroid chemistry.

“Ester” means a moiety that contains an organic moiety-C(O)—O— structure. Typically, the organic moiety-C(O)—O— structure contains about 1-50 carbon atoms (preferably 1-6 carbon atoms) and 0 to about 10 independently selected heteroatoms (e.g., O, S, N, P, Si), preferable 1 or 2 heteroatoms, where the organic moiety-C(O)—O— structure is bonded to a variable position of a steroid nucleus, replacing variable group substituents including R¹, R², R³ or R⁴ through the organic moiety-C(O)—O— structure, i.e., organic moiety-C(O)—O-steroid, preferably R¹, R² or R³. The organic moiety usually comprises one or more of any of the organic groups described herein, e.g., C₁₋₂₀ alkyl moieties (preferably C1-8), C₂₋₂₀ alkenyl moieties (preferably C2-8), C₂₋₂₀ alkynyl moieties (preferably C2-8), aryl moieties (preferably phenyl), C₁₋₉ heterocycles (preferably C2-5) or substituted derivatives of any of these, e.g., comprising 1, 2, 3, 4 or more substituents, preferably 1 or 2 substituents, preferably oxygen or nitrogen containing substituent, or halogen or optionally substituted phenyl substituents, where each substituent is independently chosen. Exemplary substitutions for hydrogen atoms in these organic groups are as described above for substituted alkyl and other substituted moieties. Substitutions are independently chosen. The organic moieties exclude obviously unstable moieties, e.g., —O—O—, except where such unstable moieties are transient species that one can use to make a compound with sufficient chemical stability for one or more of the uses described herein, including for synthesis of the formula 1 or other compounds. The substitutions listed above are typically substituents that one can use to replace a hydrogen atom, e.g., aryl, heterocycle, heteroaryl, halogen, —NH₂, —SH, —OH, alkoxy, ester, carbamate, carbonate or other functional group described herein. Preferred optionally substituted esters are acetate, enanthate, propionate, isopropionate, isobutyrate, butyrate, valerate, caproate, isocaproate, hexanoate, heptanoate, octanoate, nonanoate, decanoate, undecanoate, phenylacetate or benzoate, which are representative hydroxyl esters.

“Amide”, “amide group”, “amide moiety” and the like contains an organic moiety-C(O)—NR^{PR}- structure, where R^{PR} is —H or a protecting group, where organic moiety is as described for ester. Typically, amide groups as used here comprise an organic moiety containing about 1-50 carbon atoms (preferably C1-8) and 0 to about 10 independently selected heteroatoms (e.g., O, S, N, P, Si), preferably 1 or 2 O, S or N heteroatoms or a combination thereof. In some embodiments, the organic moiety-C(O)NR^{PR}- structure is linked to a variable position of a steroid nucleus, replacing variable group substituents that include R¹, R², R³ or R⁴, i.e., organic moiety-C(O)NR^{PR}-steroid, preferably R⁴.

“Ether” or “alkoxy group” means an organic moiety that contains 1, 2, 3, 4 or more —O— moieties, usually 1, 2 or 3, preferably 1. Typically, carbonate groups as used here comprise an organic moiety containing about 1-50 carbon atoms (preferably C1-8) and 0 to about 10 independently selected heteroatoms (e.g., O, S, N, P, Si), preferably 1 or 2 O, S or N heteroatoms or a combination thereof. In some embodiments, the organic moiety-O— structure is linked to a variable position of a steroid nucleus, replacing variable group substituents that include R¹, R², R³ or R⁴, i.e., organic moiety-O-steroid, preferably R¹, R² or R³.

“Carbonate” means an organic moiety as described for ester that comprises 1, 2, 3, 4 or more organic moiety —O—C(O)—O— structures, preferably 1. Typically, carbonate groups as used here comprise an organic moiety containing about 1-50 carbon atoms (preferably C1-8) and 0 to about 10 independently selected heteroatoms (e.g., O, S, N, P, Si), preferably 1 or 2 O, S or N heteroatoms or a combination thereof, linked to a variable position of a steroid nucleus, replacing variable group substituents that include R¹, R², R³ or R⁴ through the organic moiety —O—C(O)—O— structure, i.e., organic moiety-O—C(O)—O-steroid, preferably R¹, R² or R³.

“Carbamate” means an organic moiety that comprises 1, 2, 3, 4 or more —O—C(O)NR^{PR}-organic moiety structures where R^{PR} is —H, a protecting group or an organic moiety as described for ester. Typically, carbamate groups as used here comprise an organic moiety containing about 1-50 carbon atoms (preferably C1-8) and 0 to about 10 independently selected heteroatoms (e.g., O, S, N, P, Si), preferably 1 or 2 O, S or N heteroatoms or a combination thereof, linked to a variable position of a steroid nucleus, replacing variable group substituents that include R¹, R², R³ or R⁴ through the —O—C(O)—NR^{PR}-organic moiety structure, i.e., organic moiety-O—C(O)—NR^{PR}-steroid, preferably R¹, R² or R³.

For any group or moiety described by a given range of carbon atoms, the designated range means that any individual number of carbon atoms is described. Thus, reference to, e.g., “C1-C4 optionally substituted alkyl”, “C₂₋₆ alkenyl”, or “C2-C6 optionally substituted alkenyl”, specifically means that a 1, 2, 3 or 4 carbon optionally substituted alkyl moiety as defined herein is present, or a 2, 3, 4, 5 or 6 carbon alkenyl or optionally substituted alkenyl moiety as defined herein is present. All such designations are expressly intended to disclose all of the individual carbon atom groups and thus “C1-C4 optionally substituted alkyl” means, e.g., 3 carbon alkyl, 4 carbon substituted alkyl and the like are disclosed and can be expressly referred to or named.

“O-linked moiety”, “O-linked group” and like terms as used herein refers to an oxygen-based group or moiety that is attached to an organic moiety, directly though an oxygen atom of the oxygen-based group or moiety. An O-linked group is typically a monovalent O-linked moiety including —OH, an ester, an alkoxy group, a carbamate or a carbonate moiety as describe herein. In some embodiments the O-linked substituent is attached to the C1, C7 or C16 position (i.e. replaces R¹, R² or R³ variable group substituent) of a steroid ring system.

An “N-linked ring” means a heterocycle moiety that is bonded at a variable group position of the steroid ring system through a ring nitrogen atom of the heterocycle. The steroid ring system position for the N-linked ring substituents includes R⁴ (i.e., 17-position) substituents. N-linked rings include optionally substituted

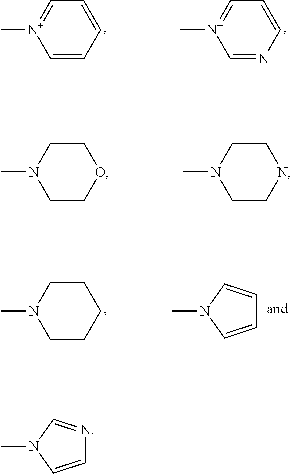

A “C-linked ring” means a heterocycle or carbocyclic ring moiety bonded at a variable group position of the steroid ring system. The rings can be saturated or unsaturated. The steroid ring system position for the C-linked ring substituent includes R⁴ (i.e., 17-position) substituents. C-linked rings include aryls and C-linked heterocycles, including C-linked heteroaryls. Exemplary C-linked rings include optionally substituted

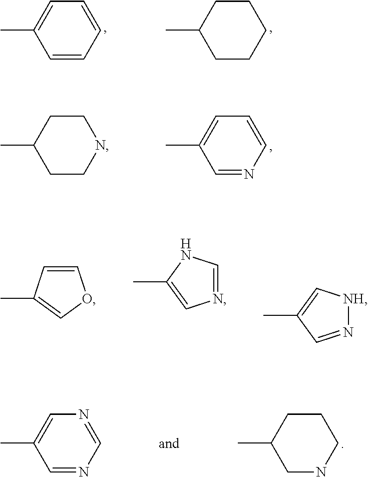

Nomenclature for the rings may vary, but will be apparent from context. For example, 3-pyridine

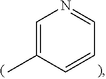

e.g., bonded to the steroid at the 17-position, may be referred to as 3-pyridyl or 3-pyridinyl. Similarly, 1-pyridinium

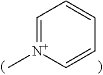

bonded to the steroid may be referred to as N-pyridyl, 1-pyridyl, N-pyridinyl or 1-pyridinyl.

Compounds that can be used in the screening method include ones having the structure

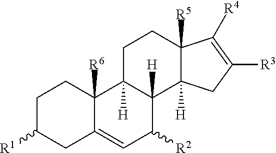

wherein, R¹ is —OH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate), an optionally substituted ether (including —O-optionally substituted C1-8 alkyl or —O-optionally substituted aryl, including —O-optionally substituted phenyl, optionally a C1-6 ether, including methoxy or ethoxy), or —SH; R² is —OH═O, an optionally substituted ester (including —O—C(O)-optionally substituted C1-7 alkyl or —O—C(O)-optionally substituted aryl, including —O-optionally substituted phenyl, optionally a C2-6 ester, including acetate or propionate, or benzoate), an optionally substituted ether (including —O-optionally substituted C1-8 alkyl or —O-optionally substituted aryl, including —O-optionally substituted phenyl, optionally a C1-6 ether, including methoxy or ethoxy), or —SH, or R² may also be —H when R³ is not —H; R³ is —H, halogen, optionally —Br, —Cl or —F, —OH, optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate), an optionally substituted ether (including —O-optionally substituted C1-8 alkyl and —O-optionally substituted aryl, including —O-optionally substituted phenyl, optionally a C1-6 ether, including methoxy or ethoxy), or C1-8 optionally substituted alkyl (including C1-4 hydroxyalkyl or C1-4 haloalkyl, optionally methyl, fluoromethyl, trifluoromethyl, ethyl, n-propyl, i-propyl, 3-fluoro-n-propyl or 3-hydroxy-n-propyl); R⁴ is -optionally substituted N-linked amide or an N-linked amino acid. Exemplary N-linked amino acids have the structure —NH—CHR⁷—C(O)OR^{PR}, -optionally substituted heterocycle or -optionally substituted cycle, including a C-linked ring (preferably a 5-membered ring or 6-membered ring) or an N-linked ring (preferably a 5-membered ring or 6-membered ring); R⁵ is —CH₃, —C₂H₅, —CH₂OH or —CH₂(ester); R⁶ is —H, —CH₃, —C₂H₅, —CH₂OH or —CH₂(ester); R⁷ is the side group of a natural amino acid (including —H, —CH₃, —CH₂OH, —CHOH—CH₃, —CH₂—CH—(CH₃)₂ or phenyl); and R^{PR} is —H or a protecting group (including a C2-6 ester optionally acetate or propionate, or benzoate), preferably —H.

Heterocycles at R⁴ include N-linked or C-linked ring moieties including N-linked or C-linked heteroaryls. Exemplary N-linked and C-linked heteroaryls include 1-furanyl, 2-furanyl, 1-oxolane, 2-oxolane, 1-thiophene, 2-thiophene, 1-pyrrole, 2-pyrrole, 3-pyrrole, 1-pyrrolidine, 2-pyrrolidine, 3-pyrrolidine, 2-thiazolyl, 3-thiazolyl, 4-thiazolyl, 5-thiazolyl, 1-pyranyl, 2-pyranyl and 3-pyranyl. Preferred heteroaryl heterocycles are 1-pyridinyl, 3-pyridinyl, 1-pyrimidinyl, 4-pyrimidinyl, and 5-pyrimidinyl.

N-linked heterocycles further include R⁴ substituents —N-pyrrolidine, —N1-pyrazolone, —N2-pyrazolone, —N-imidazolidin-2-one, —N1-imidazole, —N1-4,5-dihydroimidazole, —N-morpholine, —N1-pyridine, —N-piperidine, —N-piperazine, substituted at N4 with optionally substituted alkyl, optionally substituted aryl or optionally substuted heteroaryl, —N-indole, —N-indoline, —N-quinolidine, —NH—C(O)—CH₂—CH₂—C(O)—OH, —NH—C(O)—CH₂—C(O)—OH, —NH—C(O)—CH₂—CH₂—C(O)—OR^{PR}, —NH—C(O)—CH₂—C(O)—OR^{PR}, —NH—C(O)—(CH₂)₃—C(O)—OH or —NH—C(O)—(CH₂)₃—C(O)—OR^{PR}, wherein R^{PR} is a protecting group.

N-linked amino acids further include R⁴ substituents —NH—CH(CH₃)—C(O)OH, —NH—CH(CH₃)—C(O)OR^{PR}, —NH—CH(CH₂OH)—C(O)OH, —NH—CH(CH₂OH)—C(O)OR^{PR}, —NH—CH₂—CH₂—C(O)—OH, —NH—CH₂—C(O)—OH, —NH—CH₂—CH₂—C(O)—OR^{PR}, —NH—CH₂—C(O)—OR^{PR}, —NH—(CH₂)₃—C(O)—OH or —NH—(CH₂)₃—C(O)—OR^{PR}, wherein R^{PR} is a protecting group.

N-linked or C-linked heterocycles further include R⁴ substituents (1) —N-pyridine (N-linked) or —N-pyrimidinyl (N-linked), (2) -1-pyridyl (C-linked), -2-pyridyl, -3-pyridyl, -1-pyrimidinly (C-linked), -4-pyrimidinly or -5-pyrimidinly, (3) —N-piperidinyl, -1-piperidinyl, -2-piperidinyl, -3-piperidinyl, or (4) —N-imidazole, -2-imidazole or -4-imidazole.

Identification of compounds that reduce the level of cholesterol metabolites, particularly androgens or estrogens, are useful as agents to treat cancers, particularly neuroendocrine cancers or related cancers that grow or progress in the presence of natural androgens and/or estrogens.

Some of these compounds are new per se and can be used in the methods described herein. When the compounds are used in the screening method described herein, they are typically present as compositions containing the compound in water and/or one or more solvents of low relatively toxicity, e.g., dimethylsulfoxide, ethanol and/or methanol. Parenteral compositions for use in the screening methods in animals will typically be provided as aqueous or organic solutions or suspensions, although such compositions may or may not be suitable for human use, e.g., if significant amounts of organic solvents are present. Such compositions are most suitable for administration to animals, e.g., rodents or dogs, which can be used in the screening method. The compositions for administration to animals will typically not need to be sterile. Such compositions will typically contain about 5 mg/mL to about 50 mg/mL of the compound as a solution or suspension.

When use clinically, e.g., to treat cancer or another condition described herein, the compounds are usually presented as pharmaceutical formulations that comprise one or more excipients and the compound. Such formulations are usually prepared from purified compound that is mixed with other excipients. The compound will usually be present as a purified solid, e.g., powder or granule, that is at least about 90% w/w pure or preferably at least about 95% w/w pure, e.g., about 95% w/w to about 99.8% w/w. The formulations comprise the compound and one or more known excipients, e.g., fillers, binders, lubricants, dispersants or the like. Such excipients may include one or more of a cellulose such as microcrystalline cellulose or carboxymethylcellulose, polysorbate 80, magnesium stearate, sodium lauryl sulfate, starch or lactose. The formulations can be present as unit dosages for oral, parenteral or another other route of administration.

Unit dosages, e.g., tablets, capsules or gelcaps for oral human administration, will contain about 20 mg to about 1000 mg per unit dose, preferably about 20 mg to about 500 mg per unit dose. One or two of such unit doses are taken once per day or twice per day, e.g., twice daily. Oral dosing is preferably one or two unit dosages, most preferably one, that are taken once per day. Individual unit doses may contain about 20 mg, about 30 mg, about 50 mg, about 75 mg, about 100 mg, about 150 mg, about 200 mg, about 250 mg, about 500 mg or about 750 mg of the compound in an oral formulation.

Formulations also include those for administration by other routes including parenteral and topical including buccal or sublingual. Parenteral formulation for administration by, e.g., intravenous or intramuscular injection to patients, will be sterile solutions or, for routes other than intravenous injection, suspensions, typically aqueous. In parenteral formulations, the compound will typically be present at a concentration of about 20 mg/mL to about 100 mg/mL along with other excipients, e.g., buffers to control pH, e.g., phosphate, saline or other agents to attain roughly isotonic conditions, water, thickening agents or preservatives such as EDTA. Daily parenteral dosing will be about 10 mg/day to about 500 mg/day.

Drug products. The drug products typically comprise (a) a drug in a dosage form such as a solid or liquid formulation suitable for, e.g., oral, parenteral, topical or aerosol administration. Packaging for the drug and/or a package insert or label will have information about the drug's efficacy, mechanism of action, the intended patient population, dosage, dose regimen, route of administration, effect of the drug or treatment. When the disease to be treated is a cancer such as breast cancer or prostate cancer, the package insert or label can contain information about the patient population for which the drug product can be used or is approved.

A drug product as used herein means a product that has been reviewed and approved for marketing or sale by a regulatory agency or entity with authority to review or approve applications for sale or medical use. Uses of drug products include its marketing or sales and offers to sell or buy it for consideration. These activities will typically adhere to terms of the regulatory approval that may affect or govern marketing, sales, purchases or product handling. The drug in a drug product can be a new drug, a generic drug, a biological, a medical device or a protocol for the use of any of these. The drug product usually results from marketing approval by the U.S. Food and Drug Administration of a new drug application, an abbreviated new drug application, a biological license application or an application to market a medical device. Uses for the drug product include its sale to public or private buyers such as the U.S. Department of Defense, the U.S. Department of Energy, U.S. Department of Health and Human Services or a private drug buyer or distributor entity. Other uses include use of the drug to treat indicated or approved medical conditions and physician approved uses or off label uses.

Pre-approval drug products are other invention embodiments, which can be used, e.g., for preparing to make commercial scale product in anticipation of regulatory review or regulatory marketing approval and other drug development and review activities.

The intended patient population identified by the drug product can also specify excluded populations, if any, that may apply such as pediatric patients or elderly patients. Information about dosage will typically specify daily doses of the drug, while the dose regimen will describe how often and how long the drug is to be administered or taken. The route of administration will identify one or more routes that are suitable for use of the drug, although a given formulation will typically be approved for only one route of administration.

The compounds, e.g., as described in the claims or numbered embodiments, can be used to treat cancers such as neuroendocrine cancers such as prostate cancer or breast cancer. Cancers that can be treated include lung cancer, liver cancer and colon cancer. Cancers that can be treated further include ovarian cancer, bladder cancer and testicular. Cancers that can be treated further include endometrial cancer and cervical cancer. Cancers that can be treated further include CNS cancers such as neuroblastoma and glioma. Additional cancers that can be treated are myeloma and thyroid cancer. The compounds can also be used to treat other hormone responsive hyperproliferation conditions such as endometriosis and benign prostatic hypertrophy.

Compounds and compositions that can be used in the screening and treatment methods are described herein, e.g., in the claims and the following enumerated embodiments.

1. A compound having the structure

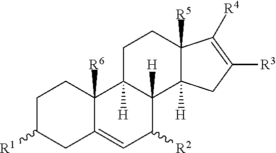

wherein, R¹ is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate), an optionally substituted ether (including —O-optionally substituted C1-8 alkyl or —O-optionally substituted aryl, including —O-optionally substituted phenyl, optionally a C1-6 ether, including —OCH₃ (methoxy ether), —OC₂H₅ (ethoxy ether), —OCH₂CH₂CH₃, —OCH₂CH₂OH, —OCH₂CH₂CH₂OH or —OCH(CH₃)₂); R² is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6,ester, including acetate or propionate, or benzoate), an optionally substituted ether (including —O-optionally substituted C1-8 alkyl or —O-optionally substituted aryl, including —O-optionally substituted phenyl, optionally a C1-6 ether, including methyl ether or ether), or optionally substituted C1-8 alkyl (including —CH₃, —CF₃, —C₂H₅, —CH₂CH₂OH, —CH₂CH₂CH₃ or —CH₂CH₂CH₂OH) or R² may also be —H when (i) R³ is not —H, (ii) R⁵ is —C₂H₅, —CH₂OH or —CH₂(ester) and/or (iii) R⁶ is —H, —C₂H₅, —CH₂OH or —CH₂(ester); R³ is —H, halogen, optionally —Br, —Cl or —F, —OH, C1-8 optionally substituted alkyl, optionally methyl, ethyl, n-propyl, i-propyl or 3-hydroxy-n-propyl, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate), an optionally substituted ether (including —O-optionally substituted C1-8 alkyl or —O-optionally substituted aryl, including —O-optionally substituted phenyl, optionally a C1-6 ether, including —OCH₃, —OC₂H₅, —OCH₂CH₂CH₃, —OCH₂CH₂OH, —OCH₂CH₂CH₂OH or —OCH(CH₃)₂), or optionally substituted C1-8 alkyl (including —CH₃, —CF₃, —C₂H₅, —CH₂CH₂OH, —CH₂CH₂CH₃, —CH₂CH₂CH₂OH or —CH(CH₃)₂); R⁴ is -optionally substituted amide, an N-linked amino acid having the structure —NH—CHR⁷—C(O)OR^{PR}, -optionally substituted heterocycle or -optionally substituted cycle, including a C-linked ring (preferably a 5-membered ring or 6-membered ring) or an N-linked ring (preferably a 5-membered ring or 6-membered ring); R⁵ is —CH₃, —C₂H₅, —CH₂OH or CH₂(ester); R⁶ is —H, —CH₃, —C₂H₅, —CH₂OH or CH₂(ester); R⁷ is the side group of a natural amino acid (including —H, —CH₃, —CH₂OH, —CHOH—CH₃, —CH₂—CH—(CH₃)₂ or phenyl); and R^{PR} is —H or a protecting group (including a C2-6 ester optionally acetate or propionate, or benzoate), preferably —H.

2. The compound of embodiment 1 wherein R⁴ is —N-pyrrolidine, —N1-pyrazolone, —N2-pyrazolone, —N-imidazolidin-2-one, —N1-imidazole, —N1-4,5-dihydroimidazole, —N-morpholine, —N1-pyridine, —N-piperidine, —N-piperazine, —N-piperazine, optionally substituted at N4 with optionally substituted alkyl or aryl, preferably methyl, phenyl or 2-pyridine, —N-indole, —N-indoline, —N-quinolidine, —NH—C(O)—CH₂—CH₂—C(O)—OH, —NH—C(O)—CH₂—C(O)—OH, —NH—C(O)—CH₂—CH₂—C(O)—OR^{PR}, —NH—C(O)—CH₂—C(O)—OR^{PR}, —NH—C(O)—(CH₂)₃—C(O)—OH or —NH—C(O)—(CH₂)₃—C(O)—OR^{PR}, wherein R^{PR} is a protecting group.

3. The compound of embodiment 1 wherein R⁴ is (1) —N-pyridine (N-linked) or —N-pyrimidinyl (N-linked), (2)-1-pyridyl (C-linked), -2-pyridyl, -3-pyridyl, -1-pyrimidinly (C-linked), -4-pyrimidinly or -5-pyrimidinly, (3) —N-piperidinyl, -1-piperidinyl, -2-piperidinyl, -3-piperidinyl, or (4) —N-imidazole, -2-imidazole or -4-imidazole.

4. The compound of embodiment 1 wherein R⁴ is 2-furanyl

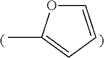

or 3-furanyl

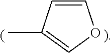

These embodiments include 17-(2-furanyl)-7β-methylandrost-5,16-diene-3β-ol, 17-(2-furanyl)-7α-methylandrost-5,16-diene-3β-ol, 17-(2-furanyl)-7α-methylandrost-5,16-diene-3α-ol, 17-(3-furanyl)-7β-ethylandrost-5,16-diene-3β-ol, 17-(3-furanyl)-7α-ethylandrost-5,16-diene-3β-ol and 17-(3-furanyl)-7β-ethylandrost-5,16-diene-3α-ol. These embodiments include analogs of these compounds where the hydrogen atom at the 16-position is substituted with an O-linked substituent, including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-furanyl)-7β-methylandrost-5,16-diene-3β,16-diol, 17-(3-furanyl)-7β-ethylandrost-5,16-diene-3β-ol-16-methyl ether and 17-(3-furanyl)-7β-ethylandrost-5,16-diene-3β-ol-16-acetate. These embodiments also include analogs of these compounds where the hydrogen atom at the 16-position is substituted with a C-linked substituent, including an optionally substituted alkyl group. Exemplary alkyl R³ substituents include —CH₃, —C₂H₅ and —CH₂CH₂CH₃ to provide exemplary species that include 17-(2-furanyl)-7β,16-dimethylandrost-5,16-diene-3β-ol and 17-(3-furanyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds further include analogs of any of these compounds where the methyl at the 19-position (R⁶) is —C₂H₅, including the analogs of the first and second named compounds in this embodiment.

5. The compound of embodiment 1 wherein R⁴ is 2-oxolane

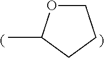

or 3-oxolane

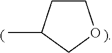

These embodiments include 17-(2-oxolanyl)-7β-methylandrost-5,16-diene-3β-ol, 17-(2-oxolanyl)-7α-methylandrost-5,16-diene-3β-ol, 17-(2-oxolanyl)-7α-methylandrost-5,16-diene-3α-ol, 17-(3-oxolanyl)-7β-ethylandrost-5,16-diene-3β-ol, 17-(3-oxolanyl)-7α-ethylandrost-5,16-diene-3β-ol and 17-(3-oxolanyl)-7β-ethylandrost-5,16-diene-3α-ol. These embodiments include analogs of these compounds where the hydrogen atom at the 16-position is substituted with an O-linked substituent, including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-oxolanyl)-7β-methylandrost-5,16-diene-3β,16-dial and 17-(3-oxolanyl)-7β-ethylandrost-5,16-diene-3β-ol-16-methyl ether. These embodiments also include analogs of these compounds where the hydrogen atom at the 16-position is substituted with carbon a C-linked substituent including an optionally substituted alkyl group. Exemplary alkyl group R³ substituents include —CH₃, —C₂H₅ and —CH₂CH₂CH₃ to provide exemplary species that include 17-(2-oxolanyl)-7β,16-dimethylandrost-5,16-diene-3β-ol and 17-(3-oxolanyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including, the analogs of the first and second named compounds in this embodiment.

6. The compound of embodiment 1 wherein R⁴ is 2-thiophene

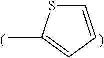

or 3-thiophene

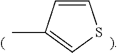

These embodiments include 17-(2-thiophenyl)-7β-methylandrost-5,16-diene-3β-ol, 17-(2-thiophenyl)-7α-methylandrost-5,16-diene-3β-ol, 17-(2-thiophenyl)-7α-methylandrost-5,16-diene-3α-ol, 17-(3-thiophenyl)-7β-ethylandrost-5,16-diene-3β-ol, 17-(3-thiophenyl)-7α-ethylandrost-5,16-diene-3β-ol and 17-(3-thiophenyl)-7β-ethylandrost-5,16-diene-3α-ol. These embodiments include analogs of these compounds where the hydrogen atom at the 16-position is substituted with an O-linked substituent including —OH, an ether or an ester. Exemplary O-linked ester and ether substituents include —OCH₃, —OC₂H₅, —OC(O)CH₃ and —OC(O)CH₂CH₃. Exemplary species of this embodiment with O-linked R³ substituents include 17-(2-thiophenyl)-7β-methylandrost-5,16-diene-3β,16-diol and 17-(3-thiophenyl)-7β-ethylandrost-5,16-diene-3β-ol-16-methyl ether. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide species that include 17-(2-thiophenyl)-7β,16-dimethylandrost-5,16-diene-3β-ol and 17-(3-thiophenyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

7. The compound of embodiment 1 wherein R⁴ is 1-pyrrole

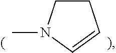

2-pyrrole

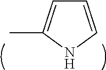

or 3-pyrrole

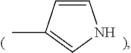

These embodiments include 17-(1-pyrrolyl)-7β-methylandrost-5,16-diene-3β-ol, 17-(1-pyrrolyl)-7α-methylandrost-5,16-diene-3β-ol, 17-(1-pyrrolyl)-7α-methylandrost-5,16-diene-3α-ol, 17-(2-pyrrolyl)-7β-methylandrost-5,16-diene-3β-ol, 17-(2-pyrrolyl)-7α-methylandrost-5,16-diene-3β-ol, 17-(2-pyrrolyl)-7α-methylandrost-5,16-diene-3α-ol, 17-(3-pyrrolyl)-7β-ethylandrost-5,16-diene-3β-ol, 17-(3-pyrrolyl)-7α-ethylandrost-5,16-diene-3β-ol and 17-(3-pyrrolyl)-7β-ethylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(1-pyrrolyl)-7β-methylandrost-5,16-diene-3β,16-diol, 17-(2-pyrrolyl)-7β-methylandrost-5,16-diene-3β,16-diol and 17-(3-pyrrolyl)-7β-ethylandrost-5,16-diene-3β-ol-16-methyl ether. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide species that include 17-(1-pyrrolyl)-7β,16-dimethylandrost-5,16-diene-3β-ol, 17-(2-pyrrolyl)-7β,16-dimethylandrost-5,16-diene-3β-ol and 17-(3-pyrrolyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment. In some of these embodiments, R⁴ is preferably 2-pyrrolyl or 3-pyrrolyl with preferred species including the 2-pyrrolyl and 3-pyrrolyl species listed above.

8. The compound of embodiment 1 wherein R⁴ is 1-pyrrolidine

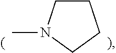

2-pyrrolidine

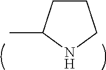

or 3-pyrrolidine

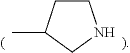

These embodiments include 17-(1-pyrrolidinyl)-7β-methylandrost-5,16-diene-3β-ol, 17-(1-pyrrolidinyl)-7α-methylandrost-5,16-diene-3β-ol, 17-(1-pyrrolidinyl)-7α-methylandrost-5,16-diene-3α-ol, 17-(2-pyrrolidinyl)-7β-methylandrost-5,16-diene-3β-ol, 17-(2-pyrrolidinyl)-7α-methylandrost-5,16-diene-3β-ol, 17-(2-pyrrolidinyl)-7α-methylandrost-5,16-diene-3α-ol, 17-(3-pyrrolidinyl)-7β-ethylandrost-5,16-diene-3β-ol, 17-(3-pyrrolidinyl)-7α-ethylandrost-5,16-diene-3β-ol and 17-(3-pyrrolidinyl)-7β-ethylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(1-pyrrolidinyl)-7β-methylandrost-5,16-diene-3β,16-diol, 17-(2-pyrrolidinyl)-7β-methylandrost-5,16-diene-3β,16-diol and 17-(3-pyrrolidinyl)-7β-ethylandrost-5,16-diene-3β-ol-16-methyl ether. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(1-pyrrolidinyl)-7β,16-dimethylandrost-5,16-diene-3β-ol, 17-(2-pyrrolidinyl)-7β,16-dimethylandrost-5,16-diene-3β-ol and 17-(3-pyrrolidinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment. In some of these embodiments R⁴ is preferably 2-pyrrolidinyl or 3-pyrrolidinyl with preferred species including the 2-pyrroldinyl and 3-pyrrolidinyl species listed above.

9. The compound of embodiment 1 wherein R⁴ is 2-thiazole

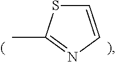

3-thiazolium

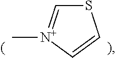

4-thiazole

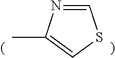

or 5-thiazole,

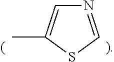

These embodiments include 17-(2-thiazolyl)-7β-methylandrost-5,16-diene-3β-ol, 17-(2-thiazolyl)-7α-methylandrost-5,16-diene-3β-ol, 17-(2-thiazolyl)-7α-methylandrost-5,16-diene-3α-ol, 17-(3-thiazolyl)-7β-ethylandrost-5,16-diene-3β-ol, 17-(3-thiazolyl)-7α-ethylandrost-5,16-diene-3β-ol, 17-(3-thiazolyl)-7β-ethylandrost-5,16-diene-3α-ol, 17-(4-thiazolyl)-7β-methylandrost-5,16-diene-3β-ol, 17-(4-thiazolyl)-7α-methylandrost-5,16-diene-3β-ol, 17-(4-thiazolyl)-7α-methylandrost-5,16-diene-3α-ol, 17-(5-thiazolyl)-7β-methylandrost-5,16-diene-3β-ol, 17-(5-thiazolyl)-7α-methylandrost-5,16-diene-3β-ol and 17-(5-thiazolyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-thiazolyl)-7β-methylandrost-5,16-diene-3β,16-diol, 17-(3-thiazolyl)-7β-methylandrost-5,16-diene-3β,16-diol, 17-(4-thiazolyl)-7β-ethylandrost-5,16-diene-3β-ol-16-methyl ether, 17-(5-thiazolyl)-7β-ethylandrost-5,16-diene-3β-ol-16-methyl ether and 17-(5-thiazolyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that includel 7-(2-thiazolyl)-7β,16-dimethylandrost-5,16-diene-3β-ol, 17-(3-thiazolyl)-7β,16-dimethylandrost-5,16-diene-3β-ol, 17-(4-thiazolyl)-7β,16-diethylandrost-5,16-diene-3β-ol and 17-(5-thiazolyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅ including the analogs of the first and second named compounds in this embodiment. In some of these embodiments, R⁴ is preferably 2-thiazolyl, 4-thiazolyl or 5-thiazolyl with preferred species including the 2-, 4- and 5-thiazolyl species listed above.

10. The compound of embodiment 1 wherein R⁴ is 2-tetrahydropyran or tetrahydropyran-2-yl

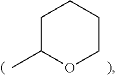

tetrahydropyran-3-yl

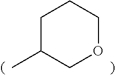

or tetrahydropyran-4-yl

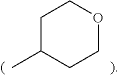

These embodiments include 17-(2-tetrahydropyranyl)-7β-methylandrost-5,16-diene-3β-ol, 17-(2-tetrahydropyranyl)-7α-methylandrost-5,16-diene-3β-ol, 17-(2-tetrahydropyranyl)-7α-methylandrost-5,16-diene-3α-ol, 17-(3-tetrahydropyranyl)-7β-ethylandrost-5,16-diene-3β-ol, 17-(3-tetrahydropyranyl)-7α-ethylandrost-5,16-diene-3β-ol, 17-(3-tetrahydropyranyl)-7β-ethylandrost-5,16-diene-3α-ol, 17-(4-tetrahydropyranyl)-7β-methylandrost-5,16-diene-3β-ol, 17-(4-tetrahydropyranyl)-7α-methylandrost-5,16-diene-3β-ol and 17-(4-tetrahydropyranyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-tetrahydropyranyl)-7β-methylandrost-5,16-diene-3β,16-diol, 17-(3-tetrahydropyranyl)-7β-methylandrost-5,16-diene-3β,16-diol and 17-(4-tetrahydropyranyl)-7β-ethylandrost-5,16-diene-3β-ol-16-methyl ether. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-tetrahydropyranyl)-7β,16-dimethylandrost-5,16-diene-3β-ol, 17-(3-tetrahydropyranyl)-7β,16-dimethylandrost-5,16-diene-3β-ol and 17-(4-tetrahydropyranyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

11. The compound of embodiment 1 wherein R⁴ is 2-(1,4-dioxane) or 1,4-dioxan-2-yl

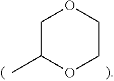

These embodiments include 17-(2-(1,4-dioxanyl))-7β-methylandrost-5,16-diene-3β-ol, 17-(2-(1,4-dioxanyl))-7α-methylandrost-5,16-diene-3β-ol and 17-(2-(1,4-dioxanyl))-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-(1,4-dioxanyl))-7β-methylandrost-5,16-diene-3β,16-dial and 17-(2-(1,4-dioxanyl))-7β-ethylandrost-5,16-diene-3β-ol-16-methyl ether. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-(1,4-dioxanyl))-7β,16-dimethylandrost-5,16-diene-3β-ol and 17-(2-(1,4-dioxanyl))-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analog of the first and second named compounds in this embodiment.

12. The compound of embodiment 1 wherein R⁴ is 2-morpholinyl or 2-morpholine

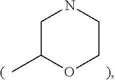

3-morpholine

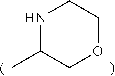

or 4-morpholine

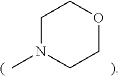

These embodiments include 17-(2-morpholinyl)-7β-methylandrost-5,16-diene-3β-ol, 17-(2-morpholinyl)-7α-methylandrost-5,16-diene-3β-ol, 17-(2-morpholinyl)-7α-methylandrost-5,16-diene-3α-ol, 17-(3-morpholinyl)-7β-ethylandrost-5,16-diene-3β-ol, 17-(3-morpholinyl)-7α-ethylandrost-5,16-diene-3β-ol, 17-(3-morpholinyl)-7β-ethylandrost-5,16-diene-3α-ol, 17-(4-morpholinyl)-7β-methylandrost-5,16-diene-3β-ol, 17-(4-morpholinyl)-7α-methylandrost-5,16-diene-3β-ol and 17-(4-morpholinyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-morpholinyl)-7β-methylandrost-5,16-diene-3β,16-diol, 17-(3-morpholinyl)-7β-methylandrost-5,16-diene-3β,16-diol, 17-(4-morpholinyl)-7β-ethylandrost-5,16-diene-3β-ol-16-methyl ether and 17-(2-morpholinyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-morpholinyl)-7β,16-dimethylandrost-5,16-diene-3β-ol, 17-(3-morpholinyl)-7β,16-dimethylandrost-5,16-diene-3β-ol and 17-(4-morpholinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment. In some of these embodiments, R⁴ is preferably 2-morpholinyl or 3-morpholinyl with preferred species including the 2- and 3-morpholinyl species listed above.

The compound of embodiment 1 wherein R⁴ is 2-oxazolyl or 2-oxazole

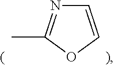

4-oxazole

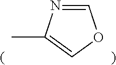

or 5-oxazole

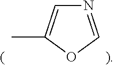

These embodiments include 17-(2-oxazolyl)-7β-methylandrost-5,16-diene-3β-ol, 17-(2-oxazolyl)-7α-methylandrost-5,16-diene-3β-ol, 17-(2-oxazolyl)-7α-methylandrost-5,16-diene-3α-ol, 17-(5-oxazolyl)-7β-ethylandrost-5,16-diene-3β-ol, 17-(5-oxazolyl)-7α-ethylandrost-5,16-diene-3β-ol, 17-(5-oxazolyl)-7β-ethylandrost-5,16-diene-3α-ol, 17-(4-oxazolyl)-7β-methylandrost-5,16-diene-3β-ol, 17-(4-oxazolyl)-7α-methylandrost-5,16-diene-3β-ol and 17-(4-oxazolyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-oxazolyl)-7β-methylandrost-5,16-diene-3β,16-diol, 17-(5-oxazolyl)-7β-methylandrost-5,16-diene-3β,16-diol or 17-(4-oxazolyl)-7β-ethylandrost-5,16-diene-3β-ol-16-methyl ether and 17-(5-oxazolyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-oxazolyl)-7β,16-dimethylandrost-5,16-diene-3β-ol, 17-(5-oxazolyl)-7β,16-dimethylandrost-5,16-diene-3β-ol and 17-(4-oxazolyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

14. The compound of embodiment 1 wherein R⁴ is 2-imidazolyl or 2-imidazole

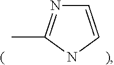

3-imidazole

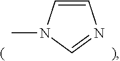

4-imidazole

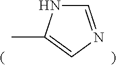

or 5-imidazole

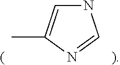

These embodiments include 17-(2-imidazolyl)-7β-methylandrost-5,16-diene-3β-ol, 17-(2-imidazolyl)-7α-methylandrost-5,16-diene-3β-ol, 17-(2-imidazolyl)-7α-methylandrost-5,16-diene-3α-ol, 17-(3-imidazolyl)-7β-ethylandrost-5,16-diene-3β-ol, 17-(3-imidazolyl)-7α-ethylandrost-5,16-diene-3β-ol, 17-(3-imidazolyl)-7β-ethylandrost-5,16-diene-3α-ol, 17-(4-imidazolyl)-7β-methylandrost-5,16-diene-3β-ol, 17-(4-imidazolyl)-7α-methylandrost-5,16-diene-3β-ol, 17-(4-imidazolyl)-7α-methylandrost-5,16-diene-3α-ol, 17-(5-imidazolyl)-7βmethylandrost-5,16-diene-3β-ol, 17-(5-imidazolyl)-7α-methylandrost-5,16-diene-3β-ol and 17-(5-imidazolyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-imidazolyl)-7β-methylandrost-5,16-diene-3β,16-diol, 17-(3-imidazolyl)-7β-methylandrost-5,16-diene-3β,16-diol, 17-(4-imidazolyl)-7β-ethylandrost-5,16-diene-3β-ol-16-methyl ether, 17-(5-imidazolyl)-7β-ethylandrost-5,16-diene-3β-ol-16-methyl ether and 17-(5-imidazolyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-imidazolyl)-7β,16-dimethylandrost-5,16-diene-3β-ol, 17-(3-imidazolyl)-7β,16-dimethylandrost-5,16-diene-3β-ol, 17-(4-imidazolyl)-7β,16-diethylandrost-5,16-diene-3β-ol and 17-(5-imidazolyl)-7β,16-diethylandrost-5,16-diene-3β-ol. These compounds also include analogs of any of these compounds where the methyl at the 18-position (R⁵) is —C₂H₅, e.g., the analog of the first and second named compounds in this embodiment. These compounds also include analogs of any of these compounds where the methyl at the 19-position (R⁶) is —C₂H₅, e.g., the analog of the first and second named compounds in this embodiment. In some of these embodiments, R⁴ is preferably 2-imidazolyl, 4-imidazolyl or 5-imidazolyl, with preferred species including the 2-, 4- and 5-imidazolyl species listed above.

15. The compound of embodiment 1 wherein R⁴ is 1-piperidinyl or 1-piperidine

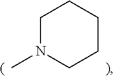

2-piperidine

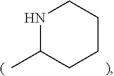

3-piperidine

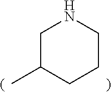

or 4-piperidine

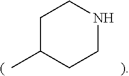

These embodiments include 17-(2-piperidinyl)-7β-methylandrost-5,16-diene-3β-ol, 17-(2-piperidinyl)-7α-methylandrost-5,16-diene-3β-ol, 17-(2-piperidinyl)-7α-methylandrost-5,16-diene-3α-ol, 17-(3-piperidinyl)-7β-ethylandrost-5,16-diene-3β-ol, 17-(3-piperidinyl)-7α-ethylandrost-5,16-diene-3β-ol, 17-(3-piperidinyl)-7β-ethylandrost-5,16-diene-3α-ol, 17-(4-piperidinyl)-7β-methylandrost-5,16-diene-3β-ol, 17-(4-piperidinyl)-7α-methylandrost-5,16-diene-3β-ol, 17-(4-piperidinyl)-7α-methylandrost-5,16-diene-3α-ol, 17-(1-piperidinyl)-7β-methylandrost-5,16-diene-3β-ol, 17-(1-piperidinyl)-7α-methylandrost-5,16-diene-3β-ol and 17-(1-piperidinyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-piperidinyl)-7β-methylandrost-5,16-diene-3β,16-diol, 17-(3-piperidinyl)-7β-methylandrost-5,16-diene-3β,16-diol, 17-(4-piperidinyl)-7β-ethylandrost-5,16-diene-3β-ol-16-methyl ether, 17-(1-piperidinyl)-7β-ethylandrost-5,16-diene-3β-ol-16-methyl ether and 17-(1-piperidinyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃, to provide exemplary species that include 17-(2-piperidinyl)-7β,16-dimethylandrost-5,16-diene-3β-ol, 17-(3-piperidinyl)-7β,16-dimethylandrost-5,16-diene-3β-ol, 17-(4-piperidinyl)-7β,16-diethylandrost-5,16-diene-3β-ol and 17-(1-piperidinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment. In some of these embodiments, R⁴ is preferably 2-piperidinyl, 3-piperidinyl or 4-piperidinyl, with preferred species including the 2-, 3- and 4-piperidinyl species listed above.

16. The compound of embodiment 1 wherein R⁴ is 1-piperazine

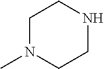

or 2-piperazine

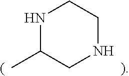

These embodiments include 17-(2-piperazinyl)-7β-methylandrost-5,16-diene-3β-ol, 17-(2-piperazinyl)-7α-methylandrost-5,16-diene-3β-ol, 17-(2-piperazinyl)-7α-methylandrost-5,16-diene-3α-ol, 17-(1-piperazinyl)-7β-ethylandrost-5,16-diene-3β-ol, 17-(1-piperazinyl)-7α-ethylandrost-5,16-diene-3β-ol and 17-(1-piperazinyl)-7β-ethylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-piperazinyl)-7β-methylandrost-5,16-diene-3β,16-diol, 17-(1-piperazinyl)-7β-ethylandrost-5,16-diene-3β-ol-16-methyl ether, 17-(1-piperazinyl)-7β-ethylandrost-5,16-diene-3β-ol-16-acetate and 17-(2-piperazinyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-piperazinyl)-7β,16-dimethylandrost-5,16-diene-3β-ol and 17-(1-piperazinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

17. The compound of embodiment 1 wherein R⁴ is 1-pyridinyl or 1-pyridinium

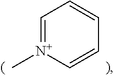

2-pryridine

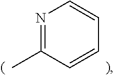

3-pyridine

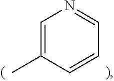

or 4-pyridine

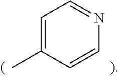

These embodiments include 17-(2-pyridinyl)-7β-methylandrost-5,16-diene-3β-ol, 17-(2-pyridinyl)-7α-methylandrost-5,16-diene-3β-ol, 17-(2-pyridinyl)-7α-methylandrost-5,16-diene-3α-ol, 17-(3-pyridinyl)-7β-ethylandrost-5,16-diene-3β-ol, 17-(3-pyridinyl)-7α-ethylandrost-5,16-diene-3β-ol, 17-(3-pyridinyl)-7β-ethylandrost-5,16-diene-3α-ol, 17-(4-pyridinyl)-7β-methylandrost-5,16-diene-3β-ol, 17-(4-pyridinyl)-7α-methylandrost-5,16-diene-3β-ol, 17-(4-pyridinyl)-7α-methylandrost-5,16-diene-3α-ol, 17-(1-pyridinyl)-7β-methylandrost-5,16-diene-3β-ol, 17-(1-pyridinyl)-7α-methylandrost-5,16-diene-3β-ol and 17-(1-pyridinyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-pyridinyl)-7β-methylandrost-5,16-diene-3β,16-diol, 17-(3-pyridinyl)-7β-methylandrost-5,16-diene-3β,16-diol, 17-(4-pyridinyl)-7β-ethylandrost-5,16-diene-3β-ol-16-methyl ether, 17-(1-pyridinyl)-7β-ethylandrost-5,16-diene-3β-ol-16-methyl ether and 17-(1-pyridinyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(2-pyridinyl)-7β,16-dimethylandrost-5,16-diene-3β-ol, 17-(3-pyridinyl)-7β,16-dimethylandrost-5,16-diene-3β-ol, 17-(4-pyridinyl)-7β,16-diethylandrost-5,16-diene-3β-ol and 17-(1-pyridinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment. In some of these embodiments, R⁴ is preferably 2-pyridinyl, 3-pyridinyl or 4-pyridinyl with preferred species including the 2-pyridinyl, 3-pyridinyl and 4-pyridinyl species listed above.

18. The compound of embodiment 1 wherein R⁴ is 1-pyrazinium

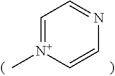

or 2-pyrazine

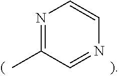

These embodiments include 17-(2-pyrazinyl)-7β-methylandrost-5,16-diene-3β-ol, 17-(2-pyrazinyl)-7α-methylandrost-5,16-diene-3β-ol, 17-(2-pyrazinyl)-7α-methylandrost-5,16-diene-3α-ol, 17-(1-pyrazinyl)-7β-ethylandrost-5,16-diene-3β-ol, 17-(1-pyrazinyl)-7α-ethylandrost-5,16-diene-3β-ol and 17-(1-pyrazinyl)-7β-ethylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-pyrazinyl)-7β-methylandrost-5,16-diene-3β,16-diol, 17-(1-pyrazinyl)-7β-ethylandrost-5,16-diene-3β-ol-16-methyl ether, 17-(1-pyrazinyl)-7β-ethylandrost-5,16-diene-3β-ol-16-acetate and 17-(2-pyrazinyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃, to provide exemplary species that include 17-(2-pyrazinyl)-7β,16-dimethylandrost-5,16-diene-3β-ol and 17-(1-pyrazinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

19. The compound of embodiment 1 wherein R⁴ is 1-pyrimidinyl or 1-pyrimidinium

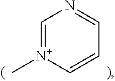

2-pyrimidine

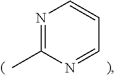

4-pyrimidine

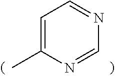

or 5-pyrimidine

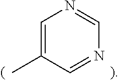

These embodiments include 17-(2-pyrimidinyl)-7β-methylandrost-5,16-diene-3β-ol, 17-(2-pyrimidinyl)-7α-methylandrost-5,16-diene-3β-ol, 17-(2-pyrimidinyl)-7α-methylandrost-5,16-diene-3α-ol, 17-(1-pyrimidinyl)-7β-ethylandrost-5,16-diene-3β-ol, 17-(1-pyrimidinyl)-7α-ethylandrost-5,16-diene-3β-ol, 17-(1-pyrimidinyl)-7β-ethylandrost-5,16-diene-3α-ol, 17-(4-pyrimidinyl)-7β-methylandrost-5,16-diene-3β-ol, 17-(4-pyrimidinyl)-7α-methylandrost-5,16-diene-3β-ol, 17-(4-pyrimidinyl)-7α-methylandrost-5,16-diene-3α-ol, 17-(5-pyrimidinyl)-7β-methylandrost-5,16-diene-3β-ol, 17-(5-pyrimidinyl)-7α-methylandrost-5,16-diene-3β-ol and 17-(5-pyrimidinyl)-7α-methylandrost-5,16-diene-3α-ol. These embodiments include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(2-pyrimidinyl)-7β-methylandrost-5,16-diene-3β,16-diol, 17-(1-pyrimidinyl)-7β-methylandrost-5,16-diene-3β,16-diol, 17-(4-pyrimidinyl)-7β-ethylandrost-5,16-diene-3β-ol-16-methyl ether, 17-(5-pyrimidinyl)-7β-ethylandrost-5,16-diene-3β-ol-16-methyl ether and 17-(5-pyrimidinyl)-7β-ethylandrost-5,16-diene-3β-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ or —CH₂CH₂CH₃ to provide exemplary species that include 17-(2-pyrimidinyl)-7β,16-dimethylandrost-5,16-diene-3β-ol, 17-(1-pyrimidinyl)-7β,16-dimethylandrost-5,16-diene-3β-ol, 17-(4-pyrimidinyl)-7β,16-diethylandrost-5,16-diene-3β-ol and 17-(5-pyrimidinyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analog of the first and second named compounds in this embodiment. In some of these embodiments, R⁴ is preferably 2-pyrimidinyl, 4-pyrimidinyl or 5-pyrimidinyl, with preferred species including the 2-pyrimidinyl, 4-pyrimidinyl and 5-pyrimidinyl species listed above.

19A-1. The compound of embodiment 1 wherein R⁴ is optionally substituted phenyl

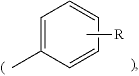

wherein R is —H, —CH₃, —C₂H₅, —CF₃, —OH, —OCH₃, —OC₂H₅ or —F. In preferred embodiments, when R is not —H, it is meta

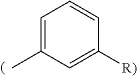

or para

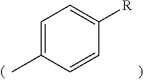

to the carbon that is bonded at the 17-position. Preferred R are —H, —CH₃ and —OCH₃. These compounds include 17-(phenyl)-7β-methylandrost-5,16-diene-3β-ol, 17-(phenyl)-7β-methylandrost-5,16-diene-3α-ol, 17-(phenyl)androst-5,16-diene-3β,7β-diol, 17-(phenyl)androst-5,16-diene-3α,7β-diol, 17-(phenyl)androst-5,16-diene-3β,7α-diol, 17-(phenyl)-7α-methylandrost-5,16-diene-3β-ol, 17-(m-methylphenyl)-7β-ethylandrost-5,16-diene-3β-ol, 17-(p-methoxyphenyl)-7β-ethylandrost-5,16-diene-3α-ol and 17-(p-fluorophenyl)-7α-ethylandrost-5,16-diene-3β-ol. These compounds include analogs of these compounds where the hydrogen atom at the 16-position is substituted with an O-linked substituent including —OH, an ether or an ester. Exemplary O-linked ester and ether R³ substituents include, —OCH₃, —OC₂H₅, —OC(O)CH₃ and —OC(O)CH₂CH₃. Exemplary species of this embodiment with O-linked R³ substituents include 17-(phenyl)-7β-methylandrost-5,16-diene-3β,16-diol, 17-(p-fluorophenyl)-7β-methylandrost-5,16-diene-3β,16-diol and 17-(p-methoxyphenyl)-7β-ethylandrost-5,16-diene-3α-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(phenyl)-7α,16-dimethylandrost-5,16-diene-3β-ol and 17-(o-hydroxyphenyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first, second and third named compounds in this embodiment.

19B. The compound of embodiment 1 wherein R⁴ is optionally substituted cyclohexyl

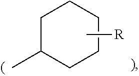

wherein R is —H, —CH₃, —C₂H₅, —CF₃, —OH, —OCH₃, —OC₂H₅ or —F. In preferred embodiments, when R is not —H, it is meta

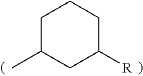

or para

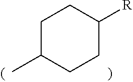

to the carbon that is bonded at the 17-position. Preferred R are —H, —F, —OCH₃ and —OH. These compounds include 17-(cyclohexyl)-7β-methylandrost-5,16-diene-3β-ol, 17-(cyclohexyl)androst-5,16-diene-3β,7β-diol, 17-(cyclohexyl)androst-5,16-diene-3α,7β-diol, 17-(p-(trifluoromethyl)cyclohexyl)-7β-methylandrost-5,16-diene-3β-ol, 17-(cyclohexyl)-7β-methylandrost-5,16-diene-3α-ol, 17-(cyclohexyl)-7α-methylandrost-5,16-diene-3β-ol, 17-(m-hydroxycyclohexyl)-7β-ethylandrost-5,16-diene-3β-ol, 17-(p-methoxycyclohexyl)-7β-ethylandrost-5,16-diene-3α-ol and 17-(p-fluorocyclohexyl)-7α-ethylandrost-5,16-diene-3β-ol. These compounds include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(cyclohexyl)-7β-methylandrost-5,16-diene-3β,16-dial and 17-(p-methoxycyclohexyl)-7β-ethylandrost-5,16-diene-3α-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(cyclohexyl)-7α,16-dimethylandrost-5,16-diene-3β-ol and 17-(o-hydroxycyclohexyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

19C. The compound of embodiment 1 wherein R⁴ is a lactone having the structure

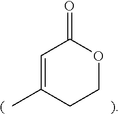

These compounds include 7β-methylandrost-5,16-diene-3β-ol-17-(pyran-3-en-2-one-3-yl), 7β-ethylandrost-5,16-diene-3β-ol-17-(pyran-3-en-2-one-3-yl), 7α-methylandrost-5,16-diene-3α-ol-17-(pyran-3-en-2-one-3-yl), 7α-ethylandrost-5,16-diene-3β-ol-17-(pyran-3-en-2-one-3-yl), androst-5,16-diene-3β,7β-diol-17-(pyran-3-en-2-one-3-yl), androst-5,16-diene-3α,7β-diol-17-(pyran-3-en-2-one-3-yl), androst-5,16-diene-3β,7α-diol-17-(pyran-3-en-2-one-3-yl) and 7α-ethylandrost-5,16-diene-3α-ol-17-(pyran-3-en-2-one-3-yl). These compounds include analogs of these compounds where the hydrogen atom at the 16-position is substituted with an O-linked substituent including —OH, an ether or an ester. Exemplary O-linked ester and ether R³ substituents include —OCH₃, —OC₂H₅, —OC(O)CH₃ and —OC(O)CH₂CH₃. Exemplary species of this embodiment with O-linked R³ substituents include 17-(cyclohexyl)-7β-methylandrost-5,16-diene-3β,16-diol and 17-(p-methoxycyclohexyl)-7β-ethylandrost-5,16-diene-3α-ol-16-acetate. These embodiments also include analogs of these compounds where the hydrogen atom at the 16-position is substituted with a C-linked substituent including an optionally substituted alkyl group. Exemplary alkyl group R³ substituents include —CH₃, —C₂H₅ and —CH₂CH₂CH₃ to provide exemplary species that include 17-(cyclohexyl)-7α,16-dimethylandrost-5,16-diene-3β-ol and 17-(o-hydroxycyclohexyl)-7β,16-diethylandrost-5,16-diene-3β-ol. These compounds also include analogs of any of these compounds where the methyl at the 18-position (R⁵) is —C₂H₅, including the analogs of the first and second named compounds in this embodiment. These compounds also include analogs of any of these compounds where the methyl at the 19-position (R⁶) is —C₂H₅, including the analogs of the first and second named compounds in this embodiment.

20. The compound of embodiment 4, 5 or 6 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂.

21. The compound of embodiment 4, 5 or 6 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —H; R³ is —H or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

22. The compound of embodiment 4, 5 or 6 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is optionally substituted C1-6 alkyl, optionally —CH₃, —CF₃, —C₂H₅, —CH₂CH₂OH, —CH₂CH₂CH₃, —CH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

23. The compound of embodiment 1, 2, 3, 20, 21 or 22 havina the structure

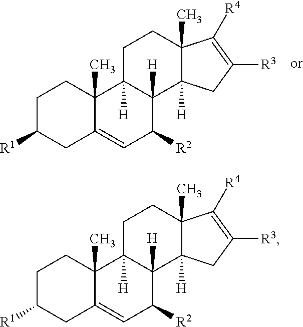

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

24. The compound of embodiment 1, 2, 3, 20, 21 or 22 having the structure

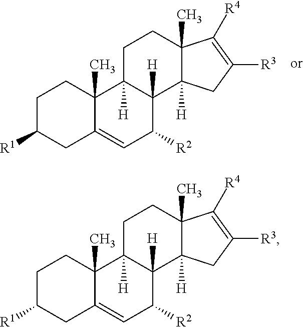

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

25. The compound of embodiment 1, 2, 3, 20, 21 or 22 having the structure

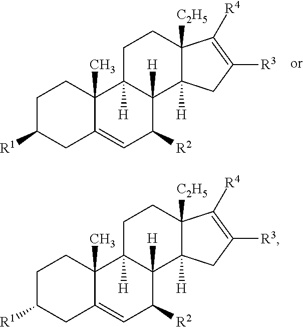

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

26. The compound of embodiment 1, 2, 3, 20, 21 or 22 having the structure

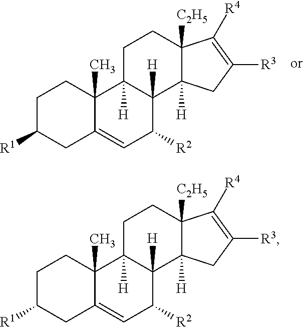

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, C₂H₅ or —CH₂CH₂OH.

27. The compound of embodiment 1, 2, 3, 20, 21 or 22 having the structure

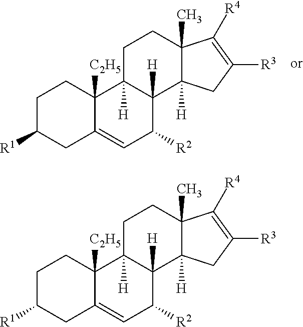

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

27. The compound of embodiment 1, 2, 3, 20, 21 or 22 having the structure

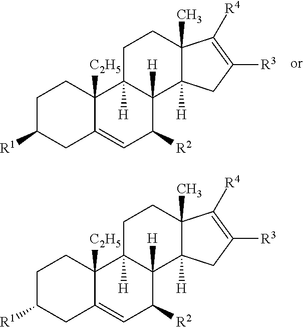

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

28. The compound of embodiment 1, 2, 3, 20, 21 or 22 having the structure

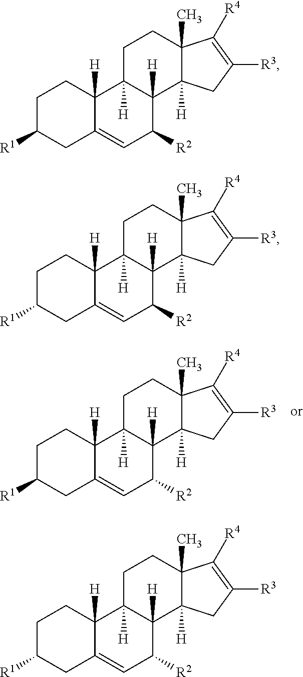

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

29. The compound of embodiment 7, 8 or 9 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂.

30. The compound of embodiment 7, 8 or 9 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —H; R³ is —H or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

31. The compound of embodiment 7, 8 or 9 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is optionally substituted C1-6 alkyl, optionally —CH₃, —CF₃, —C₂H₅, —CH₂CH₂OH, —CH₂CH₂CH₃, —CH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

32. The compound of embodiment 29, 30 or 31 having the structure

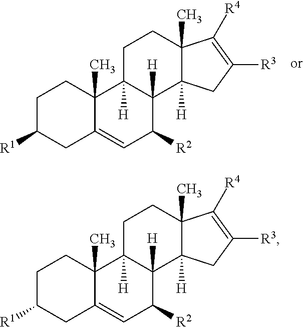

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

33. The compound of embodiment 29, 30 or 31 having the structure

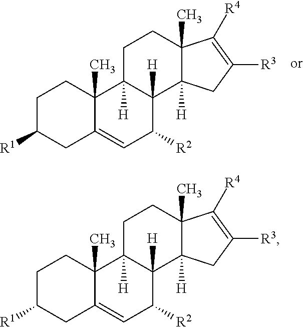

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

34. The compound of embodiment 29, 30 or 31 having the structure

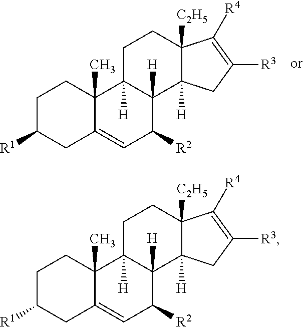

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted, preferably —CH₃, —C₂H₅ or —CH₂CH₂OH.

35. The compound of embodiment 29, 30 or 31 having the structure

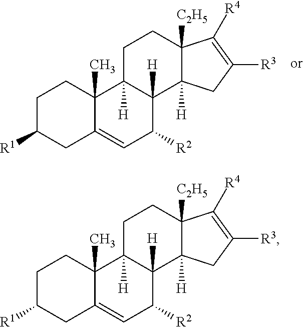

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

36. The compound of embodiment 29, 30 or 31 having the structure

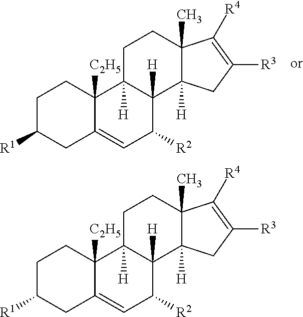

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

37. The compound of embodiment 29, 30 or 31 having the structure

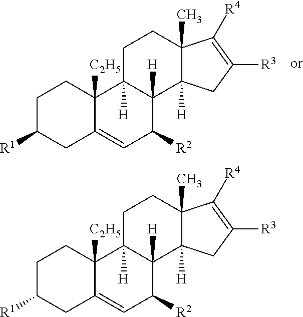

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

38. The compound of embodiment 29, 30 or 31 having the structure

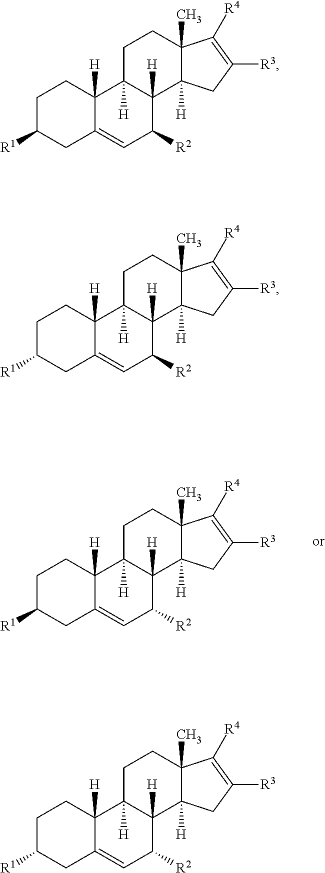

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

39. The compound of embodiment 10, 11 or 12 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂.

40. The compound of embodiment 10, 11 or 12 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —H; R³ is —H or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

41. The compound of embodiment 10, 11 or 12 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is optionally substituted C1-6 alkyl, optionally —CH₃, —CF₃, —C₂H₅, —CH₂CH₂OH, —CH₂CH₂CH₃, —CH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

42. The compound of embodiment 39, 40 or 41 having the structure

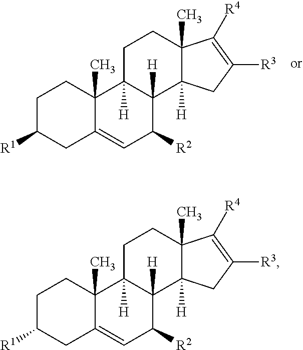

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

43. The compound of embodiment 39, 40 or 41 having the structure

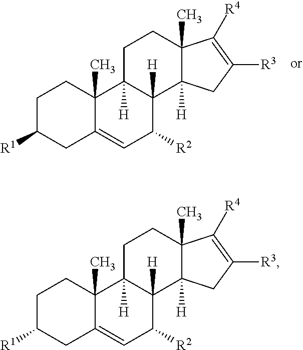

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

44. The compound of embodiment 39, 40 or 41 having the structure

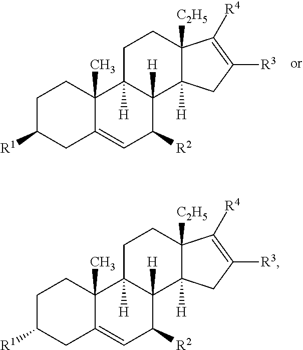

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

45. The compound of embodiment 39, 40 or 41 having the structure

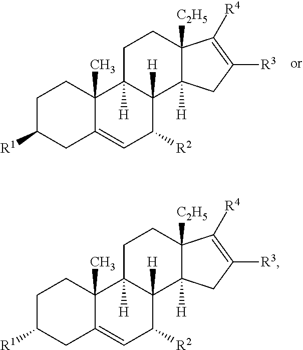

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted, preferably —CH₃, —C₂H₅ or —CH₂CH₂OH.

46. The compound of embodiment 39, 40 or 41 having the structure

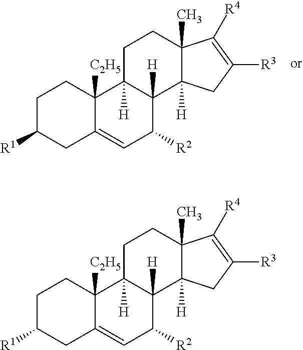

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

47. The compound of embodiment 39, 40 or 41 having the structure

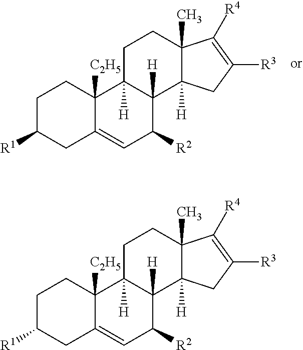

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

48. The compound of embodiment 39, 40 or 41 having the structure

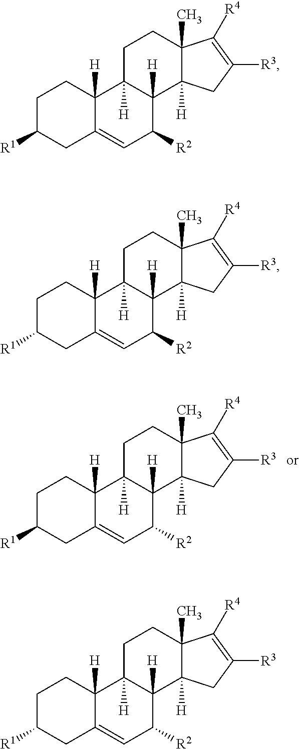

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

49. The compound of embodiment 13, 14 or 15 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂.

50. The compound of embodiment 13, 14 or 15 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —H; R³ is —H or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

51. The compound of embodiment 13, 14 or 15 wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is optionally substituted C1-6 alkyl, optionally —CH₃, —CF₃, —C₂H₅, —CH₂CH₂OH, —CH₂CH₂CH₃, —CH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

52. The compound of embodiment 49, 50 or 51 having the structure

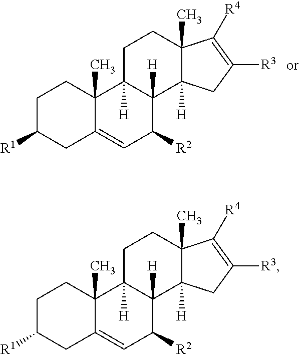

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

53. The compound of embodiment 49, 50 or 51 having the structure

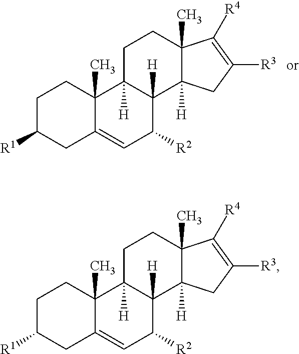

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

54. The compound of embodiment 49, 50 or 51 having the structure

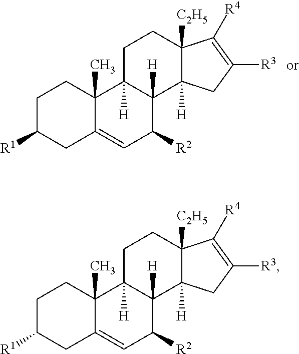

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

55. The compound of embodiment 49, 50 or 51 having the structure

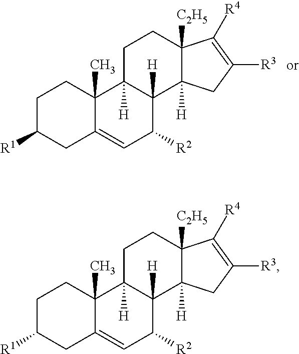

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

56. The compound of embodiment 49, 50 or 51 having the structure

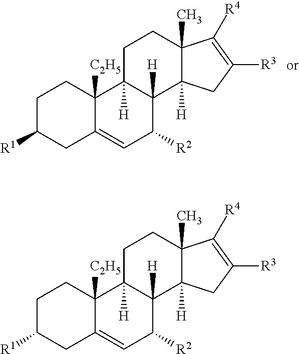

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

57. The compound of embodiment 49, 50 or 51 having the structure

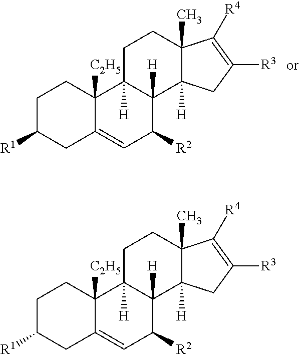

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

58. The compound of embodiment 49, 50 or 51 having the structure

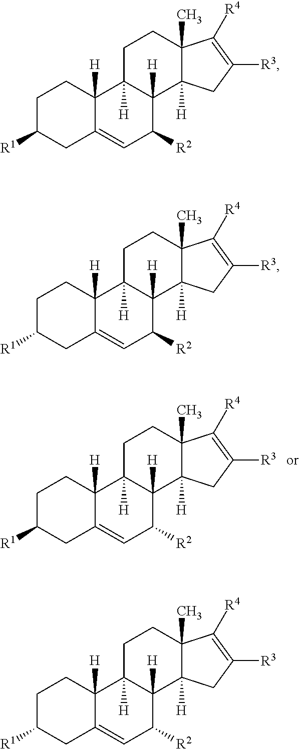

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

59. The compound of embodiment 16, 17, 18, 19, 19A-1, 19B or 19C wherein, R¹ is —OH, ═O, a C2-6 ester, optionally acetate or propionate, or a C₁₋₆ ether, optionally —OC₂H₅, —OCH₂CH₂CH₃, —OCH₂CH₂OH, —OCH₂CH₂CH₂OH or —OCH(CH₃)₂; R² is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂.

60. The compound of embodiment 16, 17, 18, 19, 19A-1, 19B or 19C wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is —H; R³ is —H or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

61. The compound of embodiment 16, 17, 18, 19, 19A-1, 19B or 19C wherein, R¹ is —OH, ═O, a C2-6 ester, optionally acetate or propionate, or a C1-6 ether, optionally —OC₂H₅, —OCH₂CH₂CH₃, —OCH₂CH₂OH, —OCH₂CH₂CH₂OH or —OCH(CH₃)₂; R² is optionally substituted C1-6 alkyl, optionally —CH₃, —CF₃, —C₂H₅, —CH₂CH₂OH, —CH₂CH₂CH₃, —CH₂CH₂CH₂OH or —OCH(CH₃)₂; R³ is —H, or C1-6 optionally substituted alkyl, optionally —CH₃, —CF₃, —C₂H₅, —CH₂CH₂OH, —CH₂CH₂CH₃, —CH₂CH₂CH₂OH or —CH(CH₃)₂. In some of these embodiments, R³ is C2-6 optionally substituted alkyl, optionally —C₂H₅, —CH₂CH₂OH, —CH₂CH₂CH₃, —CH₂CH₂CH₂OH or —CH(CH₃)₂.

62. The compound of embodiment 59, 60 or 61 having the structure

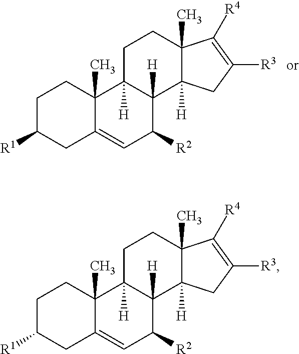

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

63. The compound of embodiment 59, 60 or 61 having the structure

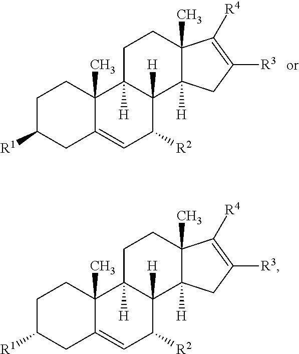

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

64. The compound of embodiment 59, 60 or 61 having the structure

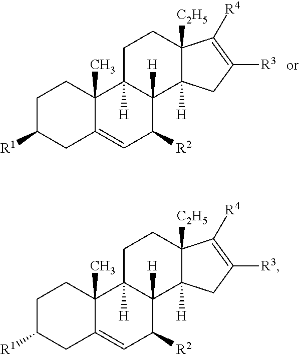

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

65. The compound of embodiment 59, 60 or 61 having the structure

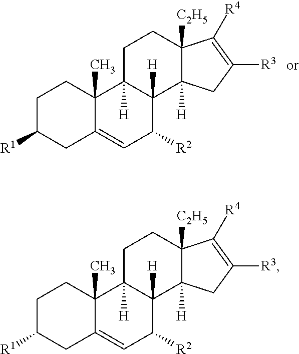

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

66. The compound of embodiment 59, 60 or 61 having the structure

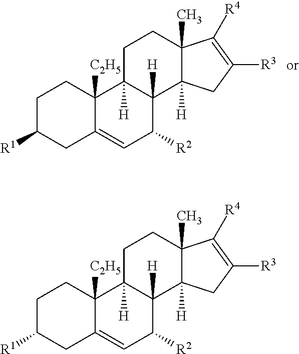

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl preferably —CH₃, —C₂H₅ or —CH₂CH₂OH.

67. The compound of embodiment 59, 60 or 61 having the structure

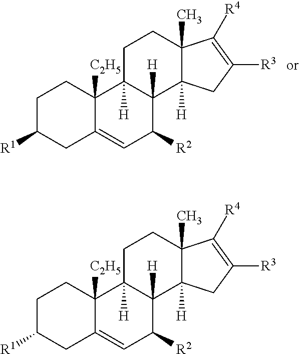

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

68. The compound of embodiment 59, 60 or 61 having the structure

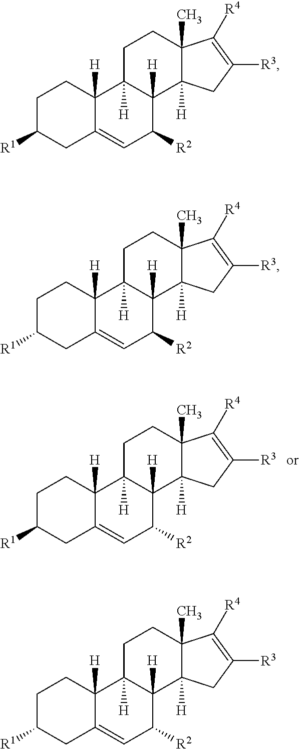

optionally wherein (a) R¹ and R² are —OH, R³ is —H, (b) R¹ is ═O, R² is —OH, R³ is —H, (c) R¹ and R² are —OH, R³ is —CH₃, (d) R¹ is ═O, R² is —OH, R³ is —CH₃, (e) R¹ is —OH, R² is —H, R³ is C1-4 optionally substituted alkyl, preferably —CH₃, —C₂H₅ or —CH₂CH₂OH.

69. The compound of embodiment 1, 2, 3, 4, 5, 6, 7, 8, 9, 10, 11, 12, 13, 14, 15, 16, 17, 18 or 19, wherein R¹ is —OH or ═O. These compounds include compounds or analogs of compounds described in embodiments 1-19.

70. The compound of embodiment 1, 2, 3, 4, 5, 6, 7, 8, 9, 10, 11, 12, 13, 14, 15, 16, 17, 18 or 19, wherein R¹ is a C2-4 ester, optionally —OC(O)CH₃ or —OC(O)CH₂CH₃. These compounds include compounds or analogs of compounds described in embodiments 1-19.

71. The compound of embodiment 1, 2, 3, 4, 5, 6, 7, 8, 9, 10, 11, 12, 13, 14, 15, 16, 17, 18, 19, 19A-1, 19B or 19C, wherein R¹ is a C1-4 ether, optionally —OCH₃ or —OCH₂CH₃. These compounds include compounds or analogs of compounds described in embodiments 1-19.

72. The compound of embodiment 69, 70 or 71 wherein R² is —H.

73. The compound of embodiment 69, 70 or 71 wherein R² is —OH.

74. The compound of embodiment 69, 70 or 71 wherein R² is a C2-4 ester, optionally —OC(O)CH₃ or —OC(O)CH₂CH₃.

75. The compound of embodiment 69, 70 or 71 wherein R² is a C1-4 ether, optionally —OCH₃ or —OCH₂CH₃.

76. The compound of embodiment 72, 73, 74 or 75 wherein R³ is —H.

77. The compound of embodiment 72, 73, 74 or 75 wherein R³ is —OH.

78. The compound of embodiment 72, 73, 74 or 75 wherein R³ is a C2-4 ester, optionally —OC(O)CH₃ or —OC(O)CH₂CH₃.

79. The compound of embodiment 72, 73, 74 or 75 wherein R³ is a C1-4 ether, optionally —OCH₃, —OCH₂CH₃, —OCH₂CH₂CH₃ or —OCH(CH₃)₂.

80. The compound of embodiment 72, 73, 74 or 75 wherein R³ is C1-4 optionally substituted alkyl, optionally —CH₃, —CH₂CH₃, —CH₂CH₂CH₃ or —CH₂CH₂OH.

81. The compound of embodiment 69, 70, 71, 72, 73, 74, 75, 76, 77, 78, 79 or 80 wherein R⁵ is —CH₃ and R⁶ is —CH₃.

82. The compound of embodiment 69, 70, 71, 72, 73, 74, 75, 76, 77, 78, 79 or 80 wherein R⁵ is —CH₂OH and R⁶ is —CH₃.

83. The compound of embodiment 69, 70, 71, 72, 73, 74, 75, 76, 77, 78, 79 or 80 wherein R⁵ is —CH₃ and R⁶ is —CH₂OH.

84. The compound of embodiment 69, 70, 71, 72, 73, 74, 75, 76, 77, 78, 79 or 80 wherein R⁵ is —CH₃ and R⁶ is —H.

85. The compound of embodiment 69, 70, 71, 72, 73, 74, 75, 76, 77, 78, 79 or 80 wherein R⁵ is —CH₂CH₃ and R⁶ is —CH₃.

86. The compound of embodiment 69, 70, 71, 72, 73, 74, 75, 76, 77, 78, 79 or 80 wherein R⁵ is —CH₂CH₃ and R⁶ is —H.

87. The compound of embodiment 69, 70, 71, 72, 73, 74, 75, 76, 77, 78, 79 or 80 wherein R⁵ is —CH₂CH₃ and R⁶ is —CH₂OH.

88. The compound of embodiment 1 wherein the compound is 17-N-pyrimidinylandrost-5,16-diene-3β,7β-diol, 17-N-pyrimidinylandrost-5,16-diene-3α,7β-diol, 17-N-pyrimidinylandrost-5,16-diene-3β,7α-diol, 17-N-pyrimidinylandrost-5,16-diene-3α,7α-diol, 17-(3-pyridyl)androst-5,16-diene-3β,7β-dial, 17-(3-pyridyl)androst-5,16-diene-3α,7β-diol, 17-(3-pyridyl)androst-5,16-diene-3β,7α-diol, 17-(3-pyridyl)androst-5,16-diene-3α,7α-diol, 17-N-pyridylandrost-5,16-diene-3β,7β-diol, 17-N-pyridylandrost-5,16-diene-3α,7β-diol, 17-N-pyridylandrost-5,16-diene-3β,7α-diol or 17-N-pyridylandrost-5,16-diene-3α,7α-diol.

89. The compound of embodiment 1 wherein the compound is 17-N-pyrimidinylandrost-5,16-diene-7β-ol-3-one, 17-N-pyrimidinylandrost-5,16-diene-7α-ol-3-one, 17-(3-pyridyl)androst-5,16-diene-7β-ol-3-one, 17-(3-pyridyl)androst-5,16-diene-7α-ol-3-one, 17-N-pyridylandrost-5,16-diene-7β-ol-3-one or 17-N-pyridylandrost-5,16-diene-7α-ol-3-one.

90. The compound of embodiment 1 wherein the compound is 17-N-pyrimidinylandrost-5,16-diene-7β-ol-3β-acetate, 17-N-pyrimidinylandrost-5,16-diene-7α-ol-3β-acetate, 17-N-pyrimidinylandrost-5,16-diene-7β-ol-3α-acetate, 17-N-pyrimidinylandrost-5,16-diene-7α-ol-3α-acetate, 17-(3-pyridyl)androst-5,16-diene-7β-ol-3β-acetate, 17-(3-pyridyl)androst-5,16-diene-7α-ol-3β-acetate, 17-N-pyridylandrost-5,16-diene-7β-ol-3β-acetate, 17-N-pyridylandrost-5,16-diene-7α-ol-3β-acetate, 17-(3-pyridyl)androst-5,16-diene-7β-ol-3α-acetate, 17-(3-pyridyl)androst-5,16-diene-7α-ol-3α-acetate, 17-N-pyridylandrost-5,16-diene-7β-ol-3α-acetate or 17-N-pyridylandrost-5,16-diene-7α-ol-3α-acetate.

91. The compound of embodiment 88, 89 or 90 wherein the compound is an analog wherein (i) the hydroxyl group at the 7-position is replaced with a C2-4 ester, optionally —O—C(O)CH₃ or —O—C(O)CH₂CH₃, e.g., 17-(3-pyridyl)androst-5,16-diene-3β-ol-7β-acetate or 17-(3-pyridyl)androst-5,16-diene-3α-ol-7β-acetate, or (ii) the hydroxyl group at the 7-position is replaced with a C2-4 ether, optionally —OCH₃ (methyl ether) or —OCH₂CH₃ (ethyl ether) with exemplary species including one or more of 17-(3-pyridyl)androst-5,16-diene-3β-ol-7β-methyl ether and 17-(3-pyridyl)androst-5,16-diene-3α-ol-7β-ethyl ether.

92. The compound of claim 91 wherein the compound is an analog wherein the hydroxyl group at the 3-position is replaced with a C3-4 ester, optionally —O—C(O)CH₂CH₃ (propionate) with exemplary species including one or more of 17-(3-pyridyl)androst-5,16-diene-7β-ol-3β-propionate and 17-(3-pyridyl)androst-5,16-diene-7β-ol-3α-propionate.

93. The compound of embodiment 1 wherein the compound is 17-N-piperidinylandrost-5,16-diene-3β,7β-diol, 17-N-piperidinylandrost-5,16-diene-3α,7β-diol, 17-N-piperidinylandrost-5,16-diene-3β,7α-diol, 17-N-piperidinylandrost-5,16-diene-3α,7α-diol, 17-(2-piperidinyl)androst-5,16-diene-3β,7β-diol, 17-(2-piperidinyl)androst-5,16-diene-3α,7β-diol, 17-(2-piperidinyl)androst-5,16-diene-3β,7α-diol, 17-(2-piperidinyl)androst-5,16-diene-3α,7α-diol, 17-(3-piperidinyl)androst-5,16-diene-3β,7β-diol, 17-(3-piperidinyl)androst-5,16-diene-3α,7β-diol, 17-(3-piperidinyl)androst-5,16-diene-3β,7α-diol or 17-(3-piperidinyl)androst-5,16-diene-3α,7α-diol.

94. The compound of embodiment 93 wherein the compound is an analog wherein the hydroxyl group at the 3-position is replaced with a C2-4 ester, optionally —O—C(O)CH₃ (acetate) or —O—C(O)CH₂CH₃ (propionate) with exemplary species including one or more of 17-(3-piperidinyl)androst-5,16-diene-7β-ol-3β-acetate, 17-(3-piperidinyl)androst-5,16-diene-7β-ol-3α-acetate and 17-(3-piperidinyl)androst-5,16-diene-7α-ol-3β-acetate.

95. The compound of embodiment 93 or 94 wherein the compound is an analog wherein (i) the hydroxyl group at the 7-position is replaced with a C2-4 ester, optionally —O—C(O)CH₃ or —O—C(O)CH₂CH₃ or (ii) the hydroxyl group at the 7-position is replaced with a C2-4 ether, optionally —OCH₃ (methyl ether) or —OCH₂CH₃ (ethyl ether), with exemplary species including one or more of 17-(3-piperidinyl)androst-5,16-diene-3β-ol-7β-ethyl ether, 17-(3-piperidinyl)androst-5,16-diene-3α-ol-7β-ethyl ether and 17-(3-piperidinyl)androst-5,16-diene-3β-ol-7α-ethyl ether.

96. The compound of embodiment 1 wherein the compound is 17-N-piperidinylandrost-5,16-diene-7β-ol-3-one, 17-N-piperidinylandrost-5,16-diene-7α-ol-3-one, 17-(2-piperidinyl)androst-5,16-diene-7β-ol-3-one, 17-(2-piperidinyl)androst-5,16-diene-7α-ol-3-one, 17-(3-piperidinyl)androst-5,16-diene-7β-ol-3-one, 17-(3-piperidinyl)androst-5,16-diene-7α-ol-3-one, 17-N-pyridinylandrost-5,16-diene-7β-ol-3-one, 17-N-pyridinylandrost-5,16-diene-7α-ol-3-one, 17-(2-pyridinyl)androst-5,16-diene-7β-ol-3-one, 17-(2-pyridinyl)androst-5,16-diene-7α-ol-3-one, 17-(3-pyridinyl)androst-5,16-diene-7β-ol-3-one or 17-(3-pyridinyl)androst-5,16-diene-7α-ol-3-one or an analog of any of these compounds wherein (i) the hydroxyl group at the 7-position is replaced with a C2-4 ester, optionally —O—C(O)CH₃ or —O—C(O)CH₂CH₃ or (ii) the hydroxyl group at the 7-position is replaced with a C2-4 ester, optionally —OCH₃ or —OCH₂CH₃.

97. The compound of claim 88, 89, 90, 91, 92, 93, 94, 95 or 96 wherein the compound is an analog wherein the hydrogen atom at the 16-position is replaced with C1-4 optionally substituted alkyl, optionally hydroxyalkyl or haloalkyl, optionally —CH₃, —CF₃, —C₂H₅, —CH₂CH₂CH₃, —CH₂CH₂CH₂CH₃, —CH₂CH₂OH, —CH₂CH₂CH₂OH or —CH₂CH₂CH₂F, with exemplary species including one or more of 17-(3-pyridyl)-16-methylandrost-5,16-diene-3β,7β-diol, 17-(3-pyridyl)-16-methylandrost-5,16-diene-3α,7β-diol, 17-(3-pyridyl)-16-methylandrost-5,16-diene-3β,7α-diol, 17-(3-pyridyl)-16-methylandrost-5,16-diene-3α,7α-diol, 17-N-pyridyl-16-methylandrost-5,16-diene-3β,7β-diol, 17-N-pyridyl-16-methylandrost-5,16-diene-3α,7β-diol, 17-N-pyridyl-16-methylandrost-5,16-diene-3β,7α-diol, 17-N-pyridyl-16-methylandrost-5,16-diene-3α,7α-diol, 17-(3-pyridyl)-16-methylandrost-5,16-diene-7β-ol-3-one, 17-(3-pyridyl)-16-methylandrost-5,16-diene-7α-ol-3-one, 17-N-pyridyl-16-methylandrost-5,16-diene-7β-ol-3-one and 17-N-pyridyl-16-methylandrost-5,16-diene-7α-ol-3-one.

98. The compound of embodiment 1 wherein the compound is 17-N-pyrimidinyl-7β-methylandrost-5,16-diene-3β-ol, 17-N-pyrimidinyl-7β-methylandrost-5,16-diene-3α-ol, 17-N-pyrimidinyl-7α-methylandrost-5,16-diene-3β-ol, 17-(2-pyrimidinyl)-7β-methylandrost-5,16-diene-3β-ol, 17-(2-pyrimidinyl)-7β-methylandrost-5,16-diene-3α-ol, 17-(2-pyrimidinyl)-7α-methylandrost-5,16-diene-3β-ol, 17-(4-pyrimidinyl)-7β-methylandrost-5,16-diene-3β-ol, 17-(4-pyrimidinyl)-7β-methylandrost-5,16-diene-3α-ol, 17-(4-pyrimidinyl)-7α-methylandrost-5,16-diene-3β-ol, 17-(5-pyrimidinyl)-7β-methylandrost-5,16-diene-3β-ol, 17-(5-pyrimidinyl)-7β-methylandrost-5,16-diene-3α-ol, 17-(5-pyrimidinyl)-7α-methylandrost-5,16-diene-3β-ol, or an analog of any of these compounds wherein the hydroxyl at the 3-position is replaced with a C2-6 ester, including acetate, with exemplary species including one or more of 17-N-pyrimidinyl-7β-methylandrost-5,16-diene-3β-acetate.

99. The compound of embodiment 1 wherein the compound is 17-(3-pyridyl)-7α-methylandrost-5,16-diene-3β-ol, 17-(3-pyridyl)-7α-ethylandrost-5,16-diene-3β-ol, 17-(3-pyridyl)-7α-methylandrost-5,16-diene-3α-ol, 17-(3-pyridyl)-7α-ethylandrost-5,16-diene-3α-ol, 17-(3-pyridyl)-7β-methylandrost-5,16-diene-3β-ol, 17-(3-pyridyl)-7β-ethylandrost-5,16-diene-3β-ol, 17-(3-pyridyl)-7β-methylandrost-5,16-diene-3α-ol, 17-(3-pyridyl)-7β-ethylandrost-5,16-diene-3α-ol, or an analog of any of these compounds wherein the hydroxyl at the 3-position is replaced with a C2-6 ester, including acetate, with exemplary species including one or more of 17-(3-pyridyl)-7α-methylandrost-5,16-diene-3β-acetate and 17-(3-pyridyl)-7α-ethylandrost-5,16-diene-3β-acetate.

100. The compound of embodiment 1 wherein the compound is 17-N-pyridyl-7β-methylandrost-5,16-diene-3β-ol, 17-N-pyridyl-7β-ethylandrost-5,16-diene-3β-ol, 17-N-pyridyl-7α-methylandrost-5,16-diene-3β-ol, 17-N-pyridyl-7α-ethylandrost-5,16-diene-3β-ol, 17-N-pyridyl-7β-methylandrost-5,16-diene-3α-ol, 17-N-pyridyl-7β-ethylandrost-5,16-diene-3α-ol, 17-N-pyridyl-7α-methylandrost-5,16-diene-3α-ol, 17-N-pyridyl-7α-ethylandrost-5,16-diene-3α-ol, or an analog of any of these compounds wherein the hydroxyl at the 3-position is replaced with a C2-6 ester, including acetate, with exemplary species including one or more of 17-N-pyridyl-7β-methylandrost-5,16-diene-3β-acetate or 17-N-pyridyl-7β-methylandrost-5,16-diene-3α-acetate.

101. The compound of embodiment 1 wherein the compound is 17-(N-imidazolyl)-7β-methylandrost-5,16-diene-3β-ol, 17-(N-imidazolyl)-7α-methylandrost-5,16-diene-3α-ol, 17-(N-imidazolyl)-7β-ethylandrost-5,16-diene-3β-ol, 17-(N-imidazolyl)-7α-ethylandrost-5,16-diene-3α-ol, 17-(N-imidazolyl)-7β-(2-hydroxyethy)landrost-5,16-diene-3β-ol, 17-(N-imidazolyl)-7α-(2-hydroxyethyl)androst-5,16-diene-3α-ol, or an analog of any of these compound wherein the hydroxyl at the 3-position is replaced with a C2-4 ester, including acetate, with exemplary species including one or more of 17-(N-imidazolyl)-7β-methylandrost-5,16-diene-3β-acetate or 17-(N-imidazolyl)-7α-(2-hydroxyethy)landrost-5,16-diene-3β-ol.

102. The compound of embodiment 1 wherein the compound is 17-(3-pyridyl)-7β-methylandrost-5,16-diene-3-one, 17-(3-pyridyl)-7β-ethylandrost-5,16-diene-3-one, 17-(3-pyridyl)-7α-methylandrost-5,16-diene-3-one, 17-(3-pyridyl)-7α-ethylandrost-5,16-diene-3-one, 17-(3-pyridyl)-7β-(2-hydroxyethypandrost-5,16-diene-3-one, 17-(3-pyridyl)-7α-(2-hydroxyethyl)androst-5,16-diene-3-one, 17-(3-pyridyl)-7β-(n-propyl)androst-5,16-diene-3-one or 17-(3-pyridyl)-7α-(n-propyl)androst-5,16-diene-3-one.

103. The compound of embodiment 1 wherein the compound is 17-N-pyrimidinyl-7β-methylandrost-5,16-diene-3-one, 17-N-pyrimidinyl-7α-methylandrost-5,16-diene-3-one, 17-N-pyrimidinyl-7β-ethylandrost-5,16-diene-3-one, 17-N-pyrimidinyl-7α-ethylandrost-5,16-diene-3-one, 17-N-pyrimidinyl-7β-(2-hydroxyethyl)androst-5,16-diene-3-one or 17-N-pyrimidinyl-7α-(2-hydroxyethyl)androst-5,16-diene-3-one.

104. The compound of embodiment 1 wherein the compound is 17-(2-pyrimidinyl)-7β-methylandrost-5,16-diene-3-one, 17-(2-pyrimidinyl)-7α-methylandrost-5,16-diene-3-one, 17-(2-pyrimidinyl)-7β-ethylandrost-5,16-diene-3-one, 17-(2-pyrimidinyl)-7α-ethylandrost-5,16-diene-3-one, 17-(2-pyrimidinyl)-7β-(2-hydroxyethyl)androst-5,16-diene-3-one or 17-(2-pyrimidinyl)-7α-(2-hydroxyethyl)androst-5,16-diene-3-one.

105. The compound of embodiment 1 wherein the compound is 17-(4-pyrimidinyl)-7β-methylandrost-5,16-diene-3-one, 17-(4-pyrimidinyl)-7α-methylandrost-5,16-diene-3-one, 17-(4-pyrimidinyl)-7β-ethylandrost-5,16-diene-3-one, 17-(4-pyrimidinyl)-7α-ethylandrost-5,16-diene-3-one, 17-(4-pyrimidinyl)-7β-(2-hydroxyethyl)androst-5,16-diene-3-one or 17-(4-pyrimidinyl)-7α-(2-hydroxyethyl)androst-5,16-diene-3-one.

106. The compound of embodiment 1 wherein the compound is 17-(N-piperidinyl)-7β-methylandrost-5,16-diene-3-one, 17-(N-piperidinyl)-7α-methylandrost-5,16-diene-3-one, 17-(N-piperidinyl)-7β-ethylandrost-5,16-diene-3-one, 17-(N-piperidinyl)-7α-ethylandrost-5,16-diene-3-one, 17-(N-piperidinyl)-7β-(2-hydroxyethyl)androst-5,16-diene-3-one or 17-(N-piperidinyl)-7α-(2-hydroxyethyl)androst-5,16-diene-3-one.

107. The compound of embodiment 1 wherein the compound is 17-(3-piperidinyl)-7β-methylandrost-5,16-diene-3-one, 17-(3-piperidinyl)-7α-methylandrost-5,16-diene-3-one, 17-(3-piperidinyl)-7β-ethylandrost-5,16-diene-3-one, 17-(3-piperidinyl)-7α-ethylandrost-5,16-diene-3-one, 17-(3-piperidinyl)-7β-(2-hydroxyethyl)androst-5,16-diene-3-one or 17-(3-piperidinyl)-7α-(2-hydroxyethyl)androst-5,16-diene-3-one.

108. The compound of embodiment 1 wherein the compound is 17-(N-piperidinyl)-7β-methylandrost-5,16-diene-3β-ol, 17-(N-piperidinyl)-7β-methylandrost-5,16-diene-3α-ol, 17-(2-piperidinyl)-7β-methylandrost-5,16-diene-3β-ol, 17-(2-piperidinyl)-7β-methylandrost-5,16-diene-3α-ol, 17-(3-piperidinyl)-7β-methylandrost-5,16-diene-3β-ol, 17-(3-piperidinyl)-7β-methylandrost-5,16-diene-3α-ol, 17-(4-piperidinyl)-7β-methylandrost-5,16-diene-3β-ol, 17-(4-piperidinyl)-7β-methylandrost-5,16-diene-3α-ol, or an analog of any of these compounds wherein the hydroxyl group at the 3-position is replaced with a C2-4 ester, optionally —O—C(O)CH₃ or —O—C(O)CH₂CH₃, with exemplary species including one or more of 17-(3-piperidinyl)-7β-metyhylandrost-5,16-diene-3β-acetate, 17-(3-piperidinyl)-7β-metyhylandrost-5,16-diene-3α-acetate and 17-(3-piperidinyl)-7β-metyhylandrost-5,16-diene-3β-acetate.

109. The compound of embodiment 88, 89, 90, 91, 92, 93, 94, 95, 96, 97, 98, 99, 100, 101, 102, 103, 104, 105, 106, 107 or 108 wherein the compound is an analog wherein the hydrogen atom at the 16-position is replaced with —OH, a C2-8 ester or a C1-8 ether with exemplary species including one or more of 17-N-pyrimidinyl-7β-methylandrost-5,16-diene-3β,16-diol, 17-N-pyrimidinyl-7β-methylandrost-5,16-diene-3α,16-diol, 17-N-pyrimidinyl-7β-methylandrost-5,16-diene-3β-ol-16-methyl ether, 17-N-pyrimidinyl-7β-methylandrost-5,16-diene-3β-ol-16-acetate, 17-(3-pyridyl)-7α-methylandrost-5,16-diene-3β,16-diol, 17-(3-pyridyl)-7α-methylandrost-5,16-diene-3β-ol-16-methyl ether, 17-(3-pyridyl)-7α-methylandrost-5,16-diene-3β-ol-16-acetate, 17-N-pyridyl-7β-ethylandrost-5,16-diene-3β-ol-16-methyl ether, 17-N-pyridyl-7β-ethylandrost-5,16-diene-3β-ol-16-ethyl ether, 17-(N-imidazolyl)-7β-methylandrost-5,16-diene-3β-ol-16-methyl ether, 17-(N-imidazolyl)-7α-(2-hydroxyethy)landrost-5,16-diene-3β-ol-16-methyl ether, 17-(3-pyridyl)-7β-methylandrost-5,16-diene-3-one-16-methyl ether, 17-(3-pyridyl)-7β-ethylandrost-5,16-diene-3-one-16-methyl ether, 17-N-pyrimidinyl-7β-methylandrost-5,16-diene-3-one-16-ethyl ether, 17-N-pyrimidinyl-7β-ethylandrost-5,16-diene-3-one-16-acetate, 17-(2-pyrimidinyl)-7β-methylandrost-5,16-diene-3-one-16-methyl ether, 17-(2-pyrimidinyl)-7β-ethylandrost-5,16-diene-3-one-16-methyl ether, 17-(4-pyrimidinyl)-7β-methylandrost-5,16-diene-3-one-16-methyl ether, 17-(4-pyrimidinyl)-7α-ethylandrost-5,16-diene-3-one-16-methyl ether, 17-(N-piperidinyl)-7β-ethylandrost-5,16-diene-3-one-16-methyl ether, 17-(N-piperidinyl)-7α-ethylandrost-5,16-diene-3-one-16-methyl ether, 17-(3-piperidinyl)-7β-ethylandrost-5,16-diene-3-one-16-methyl ether, 17-(3-piperidinyl)-7α-ethylandrost-5,16-diene-3-one-16-methyl ether, 17-(3-piperidinyl)-7β-methylandrost-5,16-diene-3β-ol-16-methyl ether and 17-(3-piperidinyl)-7β-methylandrost-5,16-diene-3α-ol-16-methyl ether.

110. Use of a compound or composition containing a compound of any preceding embodiment, including a compound or genus described or defined in any of embodiments 1-109, or a compound described in the claims for the preparation of a medicament. These compositions are used to make formulations comprising the compound and one or more excipients. Such formulations are preferably for oral or parenteral administration.

111. Use of a compound or composition containing a compound of any of embodiments 1-109 or a compound described in the claims for the preparation of a medicament for the treatment or prophylaxis of cancer. These compositions are used to make formulations comprising the compound and one or more excipients. Such formulations are preferably for oral or parenteral administration.

112. Use of a compound or composition containing a compound of any of embodiments 1-109 or a compound described in the claims for the preparation of a medicament for the treatment or prophylaxis of a neuroendocrine disorder or tumor, optionally prostate cancer, breast cancer, small cell lung cancer, a precancer of the breast, uterine fibroids, ovarian cancer, uterine cancer or endometriosis.

113. A formulation comprising one or more excipients and a compound of any of embodiments 1-109, or a compound described in the claims. In some of these embodiments, the formulation is for oral administration, including unit dosages exemplified by tablets, gelcaps or capsules, which may optionally contain amounts of structure 1 compounds that are described elsewhere herein, about 20 mg per unit dose to about 1000 mg per unit dose, including about 50 mg, about 100 mg or about 250 mg. In other embodiments, the formulation is for parenteral administration, including a sterile solution or suspension as described elsewhere herein.

114. Compounds, compositions, formulations and uses as described in any of embodiments 1-113, e.g., embodiment 52, 53, 54, 55, 93, 94, 95 or 96, wherein the double bond at the 5-position is absent and the compound has a hydrogen atom at the 5-position in the α-configuration. Exemplary compounds include 17-(3-pyridyl)-7β-methylandrost-16-ene-3-one, 17-(3-pyridyl)-7β-ethylandrost-16-ene-3-one, 17-(3-pyridyl)-7α-methylandrost-16-ene-3-one, 17-(3-pyridyl)-7α-ethylandrost-16-ene-3-one, 17-(3-pyridyl)-7β-(2-hydroxyethypandrost-16-ene-3-one, 17-(3-pyridyl)-7α-(2-hydroxyethyl)androst-16-ene-3-one, 17-(3-pyridyl)-7β-(n-propyl)androst-16-ene-3-one, 17-(3-pyridyl)-7α-(n-propyl)androst-16-ene-3-one, 17-N-pyrimidinyl-7β-methylandrost-16-ene-3-one, 17-N-pyrimidinyl-7α-methylandrost-16-ene-3-one, 17-N-pyrimidinyl-7β-ethylandrost-16-ene-3-one, 17-N-pyrimidinyl-7α-ethylandrost-16-ene-3-one, 17-N-pyrimidinyl-7β-(2-hydroxyethyl)androst-16-ene-3-one, 17-N-pyrimidinyl-7α-(2-hydroxyethyl)androst-16-ene-3-one, 17-(2-pyrimidinyl)-7β-methylandrost-16-ene-3-one, 17-(2-pyrimidinyl)-7α-methylandrost-16-ene-3-one, 17-(2-pyrimidinyl)-7β-ethylandrost-16-ene-3-one, 17-(2-pyrimidinyl)-7α-ethylandrost-16-ene-3-one, 17-(2-pyrimidinyl)-7β-(2-hydroxyethypandrost-16-ene-3-one, 17-(2-pyrimidinyl)-7α-(2-hydroxyethyl)androst-16-ene-3-one, 17-(4-pyrimidinyl)-7β-methylandrost-16-ene-3-one, 17-(4-pyrimidinyl)-7α-methylandrost-16-ene-3-one, 17-(4-pyrimidinyl)-7β-ethylandrost-16-ene-3-one, 17-(4-pyrimidinyl)-7α-ethylandrost-16-ene-3-one, 17-(4-pyrimidinyl)-7β-(2-hydroxyethypandrost-16-ene-3-one, 17-(4-pyrimidinyl)-7α-(2-hydroxyethyl)androst-16-ene-3-one, 17-(N-piperidinyl)-7β-methylandrost-16-ene-3-one, 17-(N-piperidinyl)-7α-methylandrost-16-ene-3-one, 17-(N-piperidinyl)-7β-ethylandrost-16-ene-3-one, 17-(N-piperidinyl)-7α-ethylandrost-16-ene-3-one, 17-(N-piperidinyl)-7β-(2-hydroxyethyl)androst-16-ene-3-one, 17-(N-piperidinyl)-7α-(2-hydroxyethyl)androst-16-ene-3-one, 17-(3-piperidinyl)-7β-methylandrost-16-ene-3-one, 17-(3-piperidinyl)-7α-methylandrost-16-ene-3-one, 17-(3-piperidinyl)-7β-ethylandrost-16-ene-3-one, 17-(3-piperidinyl)-7α-ethylandrost-16-ene-3-one, 17-(3-piperidinyl)-7β-(2-hydroxyethypandrost-16-ene-3-one, 17-(3-piperidinyl)-7α-(2-hydroxyethyl)androst-16-ene-3-one, 17-(N-piperidinyl)-7β-methylandrost-16-ene-3β-ol, 17-(N-piperidinyl)-7β-methylandrost-16-ene-3α-ol, 17-(2-piperidinyl)-7β-methylandrost-16-ene-3β-ol, 17-(2-piperidinyl)-7β-methylandrost-16-ene-3α-ol, 17-(3-piperidinyl)-7β-methylandrost-16-ene-3β-ol, 17-(3-piperidinyl)-7β-methylandrost-16-ene-3α-ol, 17-(4-piperidinyl)-7β-methylandrost-16-ene-3β-ol, 17-(4-piperidinyl)-7β-methylandrost-16-ene-3α-ol, or an analog of any of these compounds wherein (i) the keto group at the 3-position is replaced with —OH in the α- or β-configuration, preferably the β-configuration or (ii) the hydroxyl group at the 3-position is replaced with a C2-4 ester, optionally —O—C(O)CH₃ or —O—C(O)CH₂CH₃, with exemplary species including one or more of 17-(3-piperidinyl)-7β-methylandrost-16-ene-3β-acetate, 17-(3-piperidinyl)-7β-methylandrost-16-ene-3α-acetate and 17-(3-piperidinyl)-7β-methylandrost-16-ene-3β-acetate.

Synthesis methods. Methods A, B and C shown below can be used to prepare 17-C-heterocyclic-5,16-diene steroids and their salts. Related synthesis methods have been described, e.g., synthesis methods in US 2006-21776, US 2008-51380, U.S. Pat. No. 5,994,335, U.S. Pat. No. 5,965,548, U.S. Pat. No. 5,914,325, U.S. Pat. No. 5,677,293, U.S. Pat. No. 5,604,213, U.S. Pat. No. 5,424,304, EP 1336602, EP 721461, EP 551952, WO 09120565 and WO 0693993, which are incorporated by reference herein.

For compound 1 in methods A, B and C, R¹ is —OR^{PR}, protected hydroxyl, e.g., an ester, including —O—C(O)CH₃, —O—C(O)CH₂CH₃ or —O—C(O)CH₂CH₂CH₃, or an ether, including methyl ether or ethyl ether, R² is —OR^{PR}, protected hydroxyl, e.g., an ester, including acetate (—O—C(O)CH₃) or propionate (—O—C(O)CH₂CH₃), or an ether, including methyl ether or ethyl ether, or optionally substituted C1-8 alkyl, including —CH₃, —C₂H₅, —CH₂CH₂CH₃, —CH₂CH₂CH₂CH₃, —CH₂OR^{PR}, —CH₂CH₂OR^{PR}, —CH₂CH₂CH₂OR^{PR} or —CH₂CH₂CH₂CH₂OR^{PR}, R⁵ is —CH₃, —C₂H₅ or —CH₂OR^{PR} and R⁶ is —H, —CH₃, —C₂H₅ or —CH₂OR^{PR}, where R^{PR} is as described for R¹ and R².

Method A—Carbonyl Addition and Dehydration

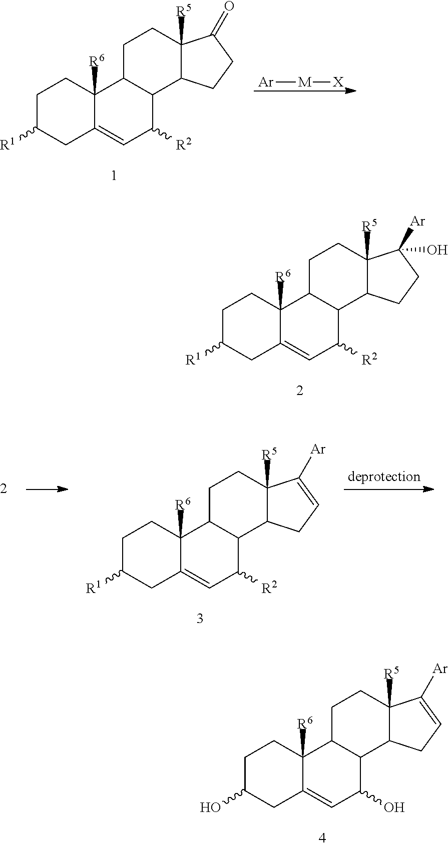

Method B—Palladium Cross Coupling

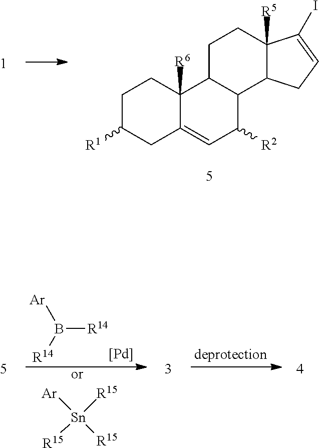

For method B, R¹⁴ are both —OH or they independently are —CH₃ or —C₂H₅. R¹⁵ independently are C1-4 saturated alkyl, including methyl or ethyl, with n-butyl preferred.

Method C—Triflate Displacement

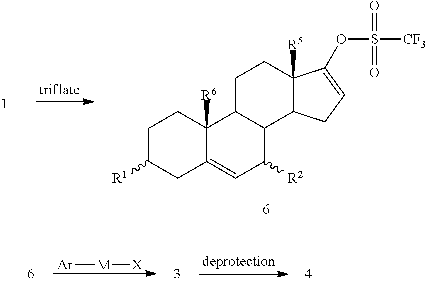

In methods A, B and C, when R¹ and R² are protected hydroxyl, e.g., an ester, including acetate, or an ether including methyl ether, ethyl ether or n-propyl ether, deprotection leaves the free hydroxyl shown as 4. When R² is alkyl, e.g., C1-6 alkyl, including methyl, ethyl, n-propyl or n-butyl, then the deprotection step results in an analog of 4 where R² is the alkyl group instead of —OH, i.e., 7.

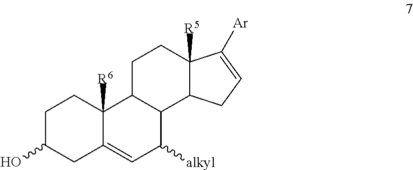

Structure 4 compounds thus include 17-(3-pyridyl)-androst-5,16-diene-3β, 7β-diol and 17-(3-pyridyl)-androst-5,16-diene-3α,7β-diol. Structure 7 compounds include 17-(3-pyridyl)-7β-(n-butypandrost-5,16-diene-3β-ol, 17-(3-pyridyl)-7β-(n-butyl)androst-5,16-diene-3α-ol, 17-(3-pyridyl)-7α-(n-butypandrost-5,16-diene-3β-ol, 17-(3-pyrimidinyl)-7β-(n-butypandrost-5,16-diene-3α-ol, 17-(3-pyrimidinyl)-7α-(n-butyl)androst-5,16-diene-3β-ol and 17-(3-pyrimidinyl)-7β-(n-butypandrost-5,16-diene-3β-ol.

As is apparent from the foregoing synthesis methods, when R¹ and R² are both in the β-configuration, compound 1 is obtained by protecting the hydroxyls at the 3- and 7-positions of precursor 7-oxodehydroepiandrosterone (androst-5-ene-3β,7β-diol-17-one), a known compound (P. Wuts et al, Organic Letters, 5:1483-1486, 2003). Similarly, when R¹ is in the α-configuration and R² is in the β-configuration, compound 1 is obtained by protecting the hydroxyls at the 3- and 7-positions of precursor androst-5-ene-3α,7β-diol-17-one, which is also a known compound.

O-linked and C-linked substituents at position-16 are introduced by one of the following methods.

Method A—Nucleophillic Epoxide Opening at C-16

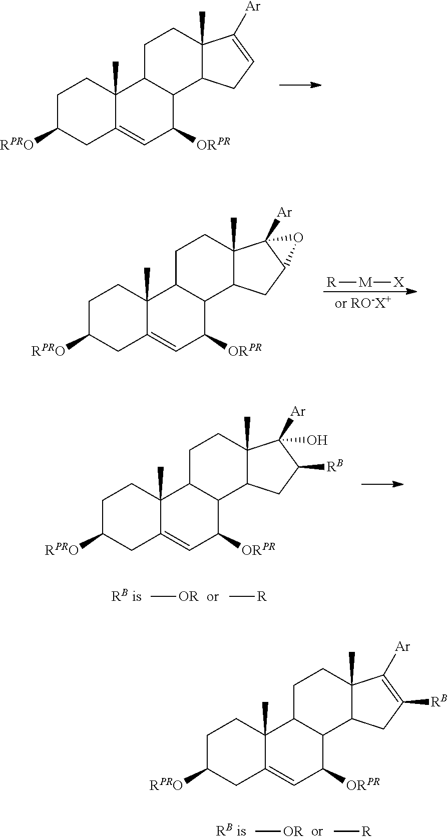

Method B—Epoxide Rearrangement to 16-one

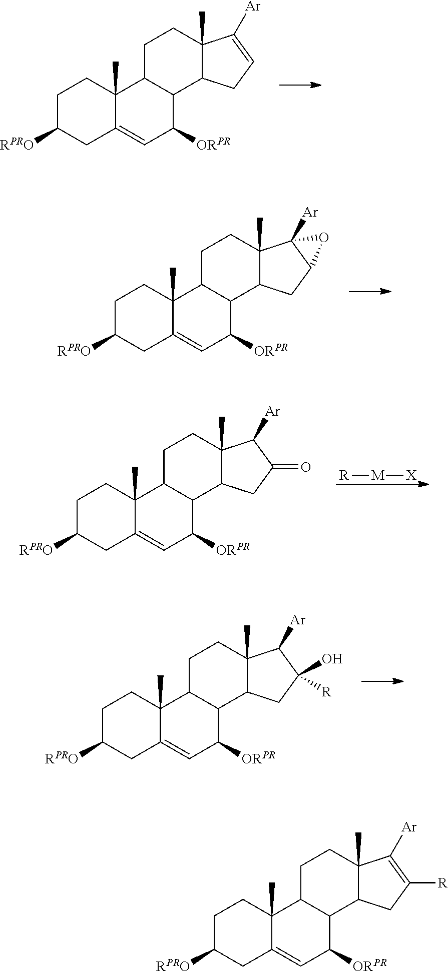

Method A and B are most suitable when Ar is substituted to provide an electron donating heterocycle in the epoxidation step. When Ar is an electron withdrawing heterocycle, protection of the Δ⁵-ene functional group may be used to optimize yields, e.g., by conversion to a C5,C6-dibromo derivative.

Other variations and modifications of the embodiments, claims and the remaining portions of this disclosure will be apparent to the skilled artisan after a reading thereof, e.g., portions on one disclosed embodiment or method can be combined with some or all of other embodiments, methods or portions of methods that are compatible therewith. Such variations and modifications are within the scope of this invention. All citations herein are incorporated herein by reference in their entirety. All citations herein are incorporated herein by reference with specificity. These citations are optionally appended to this paragraph or at new paragraphs following this paragraph.

### EXAMPLES

The following examples further illustrate the invention and they are not intended to limit it in any way. Variations and embodiments of these examples that are included in the invention include, e.g., variations of any of the examples described below as incorporated into the claims.

### Example 1

Deuterated cholesterol (D6 cholesterol) and the adrenal cell line H295R were incubated with or without the test compound, 17α-ethynylandrostane-3α,17β-diol, to observe the test compound's effects on de novo steroidogenesis in the cells. One or more of eight different cholesterol metabolites from the two metabolic pathways shown below were measured. The deuterated cholesterol was labeled at positions 2, 2, 3, 4, 4 and 6.

### Pathway 1

D6-cholesterol→D6-pregnenolone→D4-progesterone→D4-cortisol

### Pathway 2

D6-cholesterol→D6 DHEA→D6 5-androstenediol and D4 androstenedione→D4 testosterone→D4 dihydrotestosterone

The H295R cells were obtained from ATCC and grown in T75 flasks in DMEM supplemented with insulin, transferrin, selenium, and 10% FBS. The cells were grown in charcoal-stripped medium for 48 hrs prior to the experiment. Each experiment was initiated by the addition of 10 μM D6-cholesterol. After 48 hrs, the medium was removed, the cells were scraped in 5 ml methanol and the alcoholic cell suspension was lysed by sonication. The methanol lysate was dried under nitrogen and the cell contents were resuspended in 1 mL PBS. The suspension was extracted with 10 mL MTBE (methyl-t-butyl ether), which was evaporated under nitrogen. The dried extract was derivatized with nicotinyl chloride and analyzed by LCMS/MS. The cells were incubated with 300 ng/mL 17α-ethynylandrostane-3α,17β-diol. The concentration of 17α-ethynylandrostane-3α,17β-diol was monitored and adjusted to 300 ng/mL at 8 hour intervals to maintain this concentration over time.

Similar protocols can be used with radiolabeled cholesterol, e.g., cholesterol labeled with ³H or ¹⁴C.

### Example 2

Effects of the test compound 17α-ethynylandrostane-3α,17β-dial on steroidogenesis in dogs in vivo. Samples were collected from male dogs (5/group) treated with 0, 20, 60 or 200 mg/kg of 17α-ethynylandrostane-3α,17β-diol before dosing on days 1, 14, and 28. The animals were dosed daily for 28 days. The samples were analyzed for testosterone (T), androstendione (A4), and dehydroepiandrosterone (DHEA). Day 1 and day 28 vehicle and 60 mg/kg samples were assayed for luteinizing hormone. The compound was administered by oral administration of a 20 mg/mL 17α-ethynylandrostane-3α,17β-diol solution made of 40% 2-hydroxypropyl-β-cyclodextrin in water.

Plasma samples were processed by liquid/liquid extraction using MTBE. The organic portions containing the analytes were evaporated and dried extracts were incubated at 60° C. with a dansyl chloride solution. The resulting steroid derivatives were analyzed on a Waters Xbridge™ Phenyl column by reversed-phase high-performance liquid chromatography (Agilent, Palo Alto, Calif. and Leap Technologies, Carrboro, N.C.) coupled with a tandem quadrupole mass spectrometer (Waters, Beverly, Mass.). Calibration curves for standards and QC samples for HE3318 (Estradiol, E2) and Estrone (E1) were analyzed in parallel with the samples. Sample responses were acquired and concentrations were determined based on the calibration using Masslynx™ analysis software (Waters, Beverly, Mass.). Determination of QC statistics was performed by subtracting the endogenous concentration determined in native plasma from the total concentration found in the QC sample. No PK calculations were performed on this study. The quantifiable range of detection for E2 was 5.0 to 200.0 pg/mL. The quantifiable range of detection for E1 was 10.0 to 200.0 pg/mL. Values below the detection limit were identified as such and reported.

T, A4 and DHEA assay. Plasma samples were processed by liquid/liquid extraction using MTBE (methyl-t-butyl ether). The organic portions containing the free steroids were evaporated and the dried extracts were incubated at 60° C. with a hydroxylamine hydrochloride solution. The resulting steroid-oxime derivatives were extracted with MTBE. The organic portions containing the steroid-oxime derivatives were evaporated to dryness, reconstituted in 80/20 water/acetonitrile, and analyzed on a Waters Xbridge™ Phenyl column by reversed-phase high-performance liquid chromatography (Agilent, Palo Alto, Calif. and Leap Technologies, Carrboro, N.C.) coupled with a tandem quadrupole mass spectrometer (Waters, Beverly, Mass.). Calibration curves prepared in water and QC samples prepared in native plasma for T (4-androstene-3-one-17β-ol), A4 (4-androstene-3,17-dione), and DHEA were analyzed in parallel with the samples. Sample responses were acquired and concentrations were determined based on the calibration curve using Masslynx™ analysis software (Waters, Beverly, Mass.). Determination of QC statistics was performed by subtracting the endogenous concentration determined in native plasma from the total concentration found in the QC sample.

The quantifiable range of detection for T was 10.0 to 20000.0 pg/mL in rat (example 3) and dog (this example) samples. The quantifiable range of detection for A4 was 10.0 to 20000.0 pg/mL in rats and 20.0 to 20000.0 pg/mL in dogs. The quantifiable range of detection for DHEA was 50.0 to 200.0 pg/mL in both species. Values below the detection limit were identified as such.

Peptide Hormone Assays. ELISA kits for the quantification of ACTH, LH, and FSH in rat plasma were obtained from USCN Life, Wuhan, China. All other reagents were obtained from Sigma Chemical Co, St. Louis, Mo. ELISA results were measured on an ELx800 plate reader (Bio-Tek, Winooski, Vt.). Samples were assayed for ACTH, LH, and FSH concentration by means of an ELISA assay kit according to the manufacturer's instructions. For the rat samples, although 100 μL of undiluted plasma were required for each assay, 300 μL (ACTH), 100 μL (LH), or 30 μL (FSH) were used in order to obtain results in the quantifiable range of the assay. Samples from Day 14 were assayed in duplicate; samples from Day 0 and Day 7 were assayed singly due to the small sample volume available. Concentrations were calculated from an eight-point standard curve. Thirty-five μL of dog plasma was used for the LH assay.

By the end of 28 days of dosing, analysis of T and A4 in dogs indicated that levels of these hormones were decreased by about 99% compared to vehicle control treated animals. Levels of DHEA decreased by about 90% in the treated animals. Significant toxicity was not observed in the animals. The results that were obtained for testosterone are shown below.

Testosterone Concentrations

| Dose | | Conc. | Conc. | Conc. | Conc. | Conc. |
|---|---|---|---|---|---|---|
| (mg/kg) | Day | (pg/mL) | (pg/mL) | (pg/mL) | (pg/mL) | (pg/mL) |
| | | A101M | A102M | A103M | A104M | A105M |
| 0 | 1 | 444.2 | 2670.8 | 1488.3 | 2076.8 | 4223.1 |
| | 14 | 559.7 | 2768.5 | 2345.5 | 210.0 | 3123.8 |
| | 28 | 2323.5 | 1250.8 | 1228.7 | 309.7 | 4116.9 |
| | | A111M | A112M | A113M | A114M | A115M |
| 20 | 1 | 1747.2 | 1677.0 | 668.6 | 771.4 | 2450.6 |
| | 14 | 67.8 | 51.1 | 344.9 | 90.1 | 94.4 |
| | 28 | 19.3 | 60.8 | 143.8 | 173.0 | BQL |
| | | A121M | A122M | A123M | A124M | A125M |
| 60 | 1 | 46.9 | 711.2 | 723.8 | 1371.3 | 1659.6 |
| | 14 | 173.7 | 38.5 | 110.4 | 144.1 | 338.0 |
| | 28 | 18.9 | BQL | BQL | BQL | BQL |
| | | A131M | A132M | A133M | A134M | A135M |
| 200 | 1 | 503.3 | 350.3 | NS | 1816.6 | 1891.0 |
| | 14 | BQL | BQL | 119.5 | 236.6 | 18.1 |
| | 28 | BQL | NS | BQL | 11.2 | 10.2 |

Lower Limit of Detection is 10.0 pg/mL
BQL: Below the limit of quantitation
NS—No sample

### Example 3

The compound 17α-ethynylandrostane-3α,17β-diol (100 mg/kg) was administered to rats (n=5) daily for 14 days. The compound was a 20 mg/mL solution made of 30% 2-hydroxypropyl-β-cyclodextrin in water. Vehicle lacking the compound was administered to control animals (n=5). By day 7 in the dosing period, systemic (serum) levels of testosterone, estradiol, estrone and DHEA had fallen by over 99% to essentially undetectable levels in the treated animals. Changes in LH, FSH and ACTH were not observed.

To the extent not already indicated, it will be understood by those of ordinary skill in the art that any of the various specific embodiments, analysis methods, compounds or compositions described herein may be modified to incorporate other appropriate features, e.g., as shown in any other of the specific embodiments disclosed herein.

Other enumerated embodiments 1A-61A are as described below.

1A. A method to identify a compound comprising (a) administering a test compound to a mammal(s) for a sufficient period of time to obtain treated mammal(s); (b) measuring systemic levels of one or more cholesterol metabolites in the treated mammal(s); and (c) selecting the compound of step (b) that decreases the systemic levels of one or more cholesterol metabolites in the treated mammal(s), whereby a compound having a potential to treat a cancer, optionally a neuroendocrine disorder or tumor is identified, wherein the test compound of step (a) has the structure

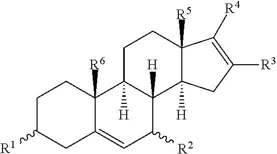

wherein, R¹ is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate) or an optionally substituted ether (—O-optionally substituted C1-8 alkyl or —O-optionally substituted aryl, optionally a C1-6 ether, including —OCH₃, —OC₂H₅, —OCH₂CH₂CH₃, —OCH₂CH₂OH, —OCH₂CH₂CH₂OH or —OCH(CH₃)₂); R² is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate) an optionally substituted ether (—O-optionally substituted C1-8 alkyl or —O-optionally substituted aryl, including —O-optionally substituted phenyl, optionally a C1-6 ether, including methoxy or ethoxy) or an optionally substituted C1-8 alkyl (including —CH₃, —CF₃, —C₂H₅, —CH₂CH₂OH, —CH₂CH₂CH₃, —CH₂CH₂CH₂OH or —OCH(CH₃)₂) or R² may also be —H when (i) R³ is not —H, (ii) R⁵ is —C₂H₅ or —CH₂OH and/or (iii) R⁶ is —H, —C₂H₅ or —CH₂OH; R³ is —H, —OH, C1-8 optionally substituted alkyl (optionally methyl, ethyl, n-propyl, i-propyl or 3-hydroxy-n-propyl), an optionally substituted ester (—O—C(O)-optionally substituted C1-7 alkyl or —O—C(O)-optionally substituted aryl, including —O—C(O)-optionally substituted phenyl, optionally a C2-6 ester such as acetate or propionate, or benzoate), an optionally substituted ether (—O-optionally substituted C1-8 alkyl or —O-optionally substituted aryl, including or —O-optionally substituted phenyl, optionally a C1-6 ether, including —OCH₃, —OC₂H₅, —OCH₂CH₂CH₃, —OCH₂CH₂OH, —OCH₂CH₂CH₂OH or —OCH(CH₃)₂) or optionally substituted C1-8 alkyl (including —CH₃, —CF₃, —C₂H₅, —CH₂CH₂OH, —CH₂CH₂CH₃, —CH₂CH₂CH₂OH or —CH(CH₃)₂); R⁴ is —NH₂, —NHCH₃, —N(CH₃)₂, —NH—C(O)CH₃, —NHOH, an N-linked amino acid, C1-8 alkyl, optionally methyl, ethyl, n-propyl or i-propyl, a C-linked ring or an N-linked ring; R⁵ is —CH₃, —C₂H₅ or —CH₂OH; and R⁶ is —H, —CH₃, —C₂H₅ or —CH₂OH.

2A. The method of embodiment 1A wherein the test compound of step (a) has the structure

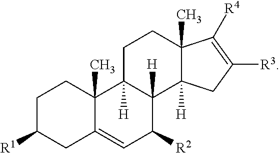

3A. The method of embodiment 1A wherein the test compound of step (a) has the structure

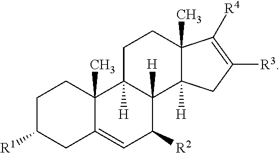

4A. The method of embodiment 1A wherein the test compound of step (a) has the structure

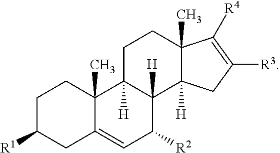

5A. The method of embodiment 1A wherein the test compound of step (a) has the structure

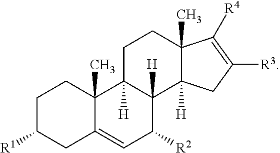

6A. The method of embodiment 1A, 2A, 3A, 4A or 5A wherein R⁴ is a C-linked ring or an N-linked ring.

7A. The method of embodiment 6A wherein R⁴ is (1) —N-pyridine (N-linked) or —N-pyrimidinyl (N-linked), (2) -1-pyridyl (C-linked), -2-pyridyl, -3-pyridyl, -1-pyrimidinly (C-linked), -4-pyrimidinly or -5-pyrimidinly, (3) —N-piperidinyl, -1-piperidinyl, -2-piperidinyl, -3-piperidinyl, or (4) —N-imidazole, -2-imidazole or -4-imidazole.

8A. The method of embodiment 1A, 2A, 3A, 4A, 5A, 6A or 7A wherein R¹ is ═O, —OH, —OC(O)CH₃, —OC(O)CH₂CH₃, —OCH₃ or —OC₂H₅.

9A. The method of embodiment 1A, 2A, 3A, 4A, 5A, 6A, 7A or 8A wherein R² is ═O, —OH, —OC(O)CH₃, —OC(O)CH₂CH₃, —OCH₃ or —OC₂H₅.

10A. The method of embodiment 1A, 2A, 3A, 4A, 5A, 6A, 7A, 8A or 9A wherein R³ is methyl, ethyl, n-propyl, i-propyl, n-butyl, sec-butyl, i-butyl or t-butyl.

11A. The method of embodiment 1A, 2A, 3A, 4A, 5A, 6A, 7A, 8A, 9A or 10A wherein the cholesterol metabolites are one or more of testosterone, dihydrotestosterone, 4-androstenedione, 5-androstenediol, estradiol, estrone, dehydroepiandrosterone, pregnenolone, progesterone and cortisol, (A) optionally wherein the cholesterol metabolites are one, two or more of (i) testosterone, dihydrotestosterone, 4-androstenedione, 5-androstenediol, 5α-androstane-3α-17β-diol or 5α-androstane-3β-17β-diol (ii) estradiol, estrone and 4-androstenedione or (iii) pregnenolone, progesterone and cortisol, (B) optionally wherein the cholesterol metabolites are (a) one, two or more of testosterone, dihydrotestosterone, 4-androstenedione, 5-androstenediol or (b) one or both of estradiol and estrone.

12A. The method of embodiment 1A, 2A, 3A, 4A, 5A, 6A, 7A, 8A, 9A, 10A or 11A wherein the sufficient period of time is at least about 5 days, optionally about 5 days to about 8 weeks, optionally daily for about 7 days, about 14 days, about 28 days, about 6 weeks or about 8 weeks, e.g., daily for 5 days, 7 days, 14 days, 28 days, 6 weeks or 8 weeks.

13A. The method of embodiment 1A, 2A, 3A, 4A, 5A, 6A, 7A, 8A, 9A, 10A or 11A wherein the neuroendocrine disorder or tumor is prostate cancer, breast cancer or small cell lung cancer and the candidate compound is administered to a human(s) having or diagnosed with, the neuroendocrine disorder or tumor.

14A. The method of embodiment 1A, 2A, 3A, 4A, 5A, 6A, 7A, 8A, 9A, 10A or 11A wherein the neuroendocrine disorder or tumor is a precancer of the breast, uterine fibroids, ovarian cancer, uterine cancer or endometriosis and the candidate compound is administered to a human(s) having the neuroendocrine disorder or tumor.

15A. The method of embodiment 1A, 2A, 3A, 4A, 5A, 6A, 7A, 8A, 9A, 10A or 11A wherein the neuroendocrine disorder or tumor is an adrenal tumor, benign prostatic hypertrophy or testicular cancer and the candidate compound is administered to a human(s) having the neuroendocrine disorder or tumor.

16A. The method of embodiment 1A, 2A, 3A, 4A, 5A, 6A, 7A, 8A, 9A, 10A, 11A or 12A wherein the mammal(s) is a canine (dog) or a rodent, optionally a mouse or rat.

17A. The method of embodiment 1A, 2A, 3A, 4A, 5A, 6A, 7A, 8A, 9A, 10A, 11A or 12A further comprising administering to a control mammal(s) a control compound, optionally, 17α-ethynylandrostane-3α,17β-diol, 17α-ethynylandrostane-3β,17β-diol, 17α-ethynylandrostane-3-one-17β-ol or an aromatase inhibitor and measuring systemic levels of the one or more cholesterol metabolites in the treated mammal(s).

18A. A method to make a drug product for treating a cancer or neuroendocrine disorder or tumor in a human, wherein the drug product comprises, (a) a drug in a dosage form, optionally wherein the dosage form is a formulation for oral, parenteral or topical administration, preferably oral administration; and (b) packaging for the drug together with a package insert or label that includes information about the drug's efficacy, toxicity or mechanism of action wherein such information was obtained at least in part from a method comprising (i) administering a test compound to a mammal(s) for a sufficient period of time to obtain treated mammal(s); (ii) measuring systemic levels of one or more cholesterol metabolites in the treated mammal(s); (iii) selecting the compound of step (ii) that decreases the systemic levels of one or more cholesterol metabolites in the treated mammal(s); and optionally (iv) administering to a control mammal(s) a control compound, optionally, 17α-ethynylandrostane-3α,17β-diol, 17α-ethynylandrostane-3β,17β-diol, 17α-ethynylandrostane-3-one-17β-ol or an aromatase inhibitor and measuring systemic levels of the one or more cholesterol metabolites in the mammal(s) and optionally comparing the effect of the control compound on the treated mammals with the effect of the test compound on the treated mammals, whereby a compound having a potential to treat a neuroendocrine disorder or tumor is identified, wherein the test compound of step (a) has the structure defined in embodiment 1.

19A. The drug product of embodiment 18A wherein the mammal(s) is a rodent(s) or canine(s), optionally a mouse or rat.

20A. The drug product of embodiment 18A or 19A wherein the neuroendocrine disorder or tumor is prostate cancer, breast cancer or small cell lung cancer.

21A. The drug product of embodiment 18A or 19A wherein the neuroendocrine disorder or tumor is a precancer of the breast, uterine fibroids, ovarian cancer, uterine cancer or endometriosis.

22A. The drug product of embodiment 18A or 19A wherein the neuroendocrine disorder or tumor is an adrenal tumor, benign prostatic hypertrophy or testicular cancer.

23A. A compound having the structure

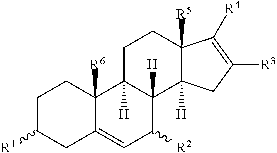

wherein, R¹ is —OH, —SH, ═O, an optionally substituted ester (including —O—C(O)-optionally substituted C1-7 alkyl or —O—C(O)-optionally substituted aryl, including —O—C(O)-optionally substituted phenyl, optionally a C2-6 ester, including acetate or propionate, or benzoate) or an optionally substituted ether (—O-optionally substituted C1-8 alkyl or —O-optionally substituted C1-8 aryl, including —O-optionally substituted phenyl, optionally a C1-6, including-OCH₃, —OC₂H₅, —OCH₂CH₂CH₃, —OCH₂CH₂OH, —OCH₂CH₂CH₂OH or —OCH(CH₃)₂); R² is —OH, —SH, ═O, an optionally substituted ester (—O—C(O)-optionally substituted C1-7 alkyl or —O—C(O)-optionally substituted aryl, including —O—C(O)-optionally substituted phenyl, optionally a C2-6 ester, including acetate or propionate, or benzoate), an optionally substituted ether (—O-optionally substituted C1-8 alkyl or —O-optionally substituted aryl, including —O-optionally substituted phenyl, optionally a C1-6 ether, including methoxy or ethoxy), or an optionally substituted C1-8 alkyl (including —CH₃, —CF₃, —C₂H₅, —CH₂CH₂OH, —CH₂CH₂CH₃, —CH₂CH₂CH₂OH or —OCH(CH₃)₂) or R² may also be —H when (i) R³ is not —H, (ii) R⁵ is —C₂H₅ or —CH₂OH and/or (iii) R⁶ is —H, —C₂H₅ or —CH₂OH; R³ is —H, —OH, C1-8 optionally substituted alkyl (optionally methyl, ethyl, n-propyl, i-propyl or 3-hydroxy-n-propyl), an ester (including —O—C(O)-optionally substituted C1-7 alkyl or —O—C(O)-optionally substituted aryl, including —O—C(O)-optionally substituted C1-7 phenyl, optionally a C2-6 ester, including acetate or propionate, or benzoate), an optionally substituted ether (including —O-optionally substituted C1-8 alkyl or —O-optionally substituted aryl, including —O-optionally substituted phenyl, optionally a C1-6 ether, including —OCH₃, —OC₂H₅, —OCH₂CH₂CH₃, —OCH₂CH₂OH, —OCH₂CH₂CH₂OH or —OCH(CH₃)₂) or an optionally substituted C1-8 alkyl (including —CH₃, —CF₃, —C₂H₅, —CH₂CH₂OH, —CH₂CH₂CH₃, —CH₂CH₂CH₂OH or —CH(CH₃)₂); R⁴ is an optionally substituted heterocycle or optionally substituted cycle, wherein the heterocycle or cycle is a C-linked ring (bonded to the 17-position through a ring carbon), preferably a 5-membered ring or 6-membered ring; R⁵ is —CH₃, —C₂H₅ or —CH₂OH; and R⁶ is —H, —CH₃, —C₂H₅ or —CH₂OH.

24A. The compound of embodiment 23A wherein R⁴ is 2-pyridyl, 3-pyridyl or 4-pyridyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

25A. The compound of embodiment 23A wherein R⁴ is 2-pyrimidinlyl, 4-pyrimidinly or 5-pyrimidinly, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

26A. The compound of embodiment 23A wherein R⁴ is 2-piperidinyl, 3-piperidinyl or 4-piperidinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

27A. The compound of embodiment 23A wherein R⁴ is 2-imidazole or 4-imidazole, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

28A. The compound of embodiment 23A wherein R⁴ is 2-furanyl or 3-furanyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

29A. The compound of embodiment 23A wherein R⁴ is 2-oxolanyl or 3-oxolanyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

30A. The compound of embodiment 23A wherein R⁴ is 2-thiophenyl or 3-thiophenyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

31A. The compound of embodiment 23A wherein R⁴ is 2-pyrrolyl or 3-pyrrolyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

32A. The compound of embodiment 23A wherein R⁴ is 2-pyrrolidinyl or 3-pyrrolidinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

33A. The compound of embodiment 23A wherein R⁴ is 2-thiazolyl, 4-thiazolyl or 5-thiazolyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

34A. The compound of embodiment 23A wherein R⁴ is 2-oxolanyl (2-tetrahydropyranyl), 3-oxolanyl or 4-oxolanyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

35A. The compound of embodiment 23A wherein R⁴ is 2-(1,4-dioxanyl), optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

36A. The compound of embodiment 23A wherein R⁴ is 2-morpholinyl or 3-morpholinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

37A. The compound of embodiment 23A wherein R⁴ is 2-oxazolyl, 4-oxazolyl or 5-oxazolyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

38A. The compound of embodiment 23A wherein R⁴ is 2-imidazolyl, 4-imidazolyl or 5-imidazolyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

39A. The compound of embodiment 23A wherein R⁴ is 2-piperidinyl, 3-piperidinyl or 4-piperidinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

40A. The compound of embodiment 23A wherein R⁴ is 2-piperazinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

41A. The compound of embodiment 23A wherein R⁴ is 2-pyridinyl, 3-pyridinyl or 4-pyridinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

42A. The compound of embodiment 23A wherein R⁴ is 2-pyrazinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

43A. The compound of embodiment 23A wherein R⁴ is 2-pyrimidinyl, 4-pyrimidinyl or 5-pyrimidinyl, optionally wherein (i) R¹ and R² are —OH in the β-configuration, R⁵ and R⁶ are —CH₃ and R³ is —CH₃ or (ii) R¹ is ═O, R² is —OH in the β-configuration, R⁵ and R⁶ are —CH₃ and R³ is —H.

44A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

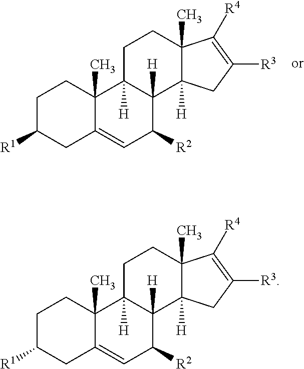

45A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

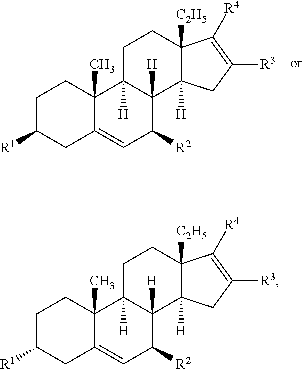

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl, including —CH₃, —C₂H₅ or —CH₂CH₂OH, or optionally wherein the compound is an analog of a compound named in enumerated embodiment 4A, 5A, 6A 7A, 8A, 9A, 10A, 11A, 12A, 13A, 14A, 15A, 16A, 17A, 18A or 19A, wherein in the analog, R⁵ is —C₂H₅, including species 7-(3-pyridinyl)-18-nor-18-ethylandrost-5,16-diene-3β,7β-diol, which is

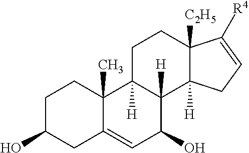

where R⁴ is 3-pyridinyl, 7-(3-pyridinyl)-18-nor-18-ethylandrost-5,16-diene-3α,7β-diol or 7-(3-pyridinyl)-18-nor-18-ethylandrost-5,16-diene-3β,7β-diol-16-acetate.

46A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

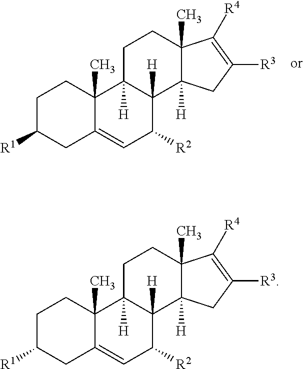

47A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

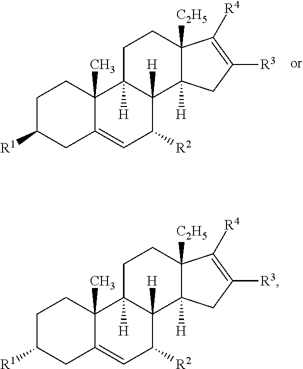

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl, including —C₂H₅ or —CH₂CH₂OH, or optionally wherein the compound is an analog of a compound named in enumerated embodiment 4A, 5A, 6A 7A, 8A, 9A, 10A, 11A, 12A, 13A, 14A, 15A, 16A, 17A, 18A or 19A, wherein in the analog, R⁵ is —C₂H₅, including species 17-(3-pyridinyl)-18-nor-18-ethylandrost-5,16-diene-3β,7α-diol, which is

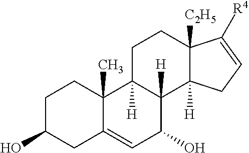

where R⁴ is 3-pyridinyl or 17-(3-pyridinyl)-18-nor-18-ethylandrost-5,16-diene-3α,7α-diol or 17-(3-pyridinyl)-18-nor-18-ethylandrost-5,16-diene-3β,7α-diol-16-acetate.

48A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

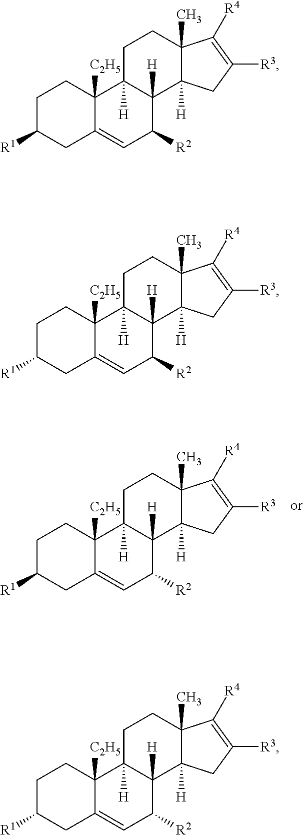

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl, including —CH₃, —C₂H₅ or —CH₂CH₂OH, or optionally wherein the compound is an analog of a compound named in enumerated embodiment 4A, 5A, 6A 7A, 8A, 9A, 10A, 11A, 12A, 13A, 14A, 15A, 16A, 17A, 18A or 19A, wherein in the analog, R⁶ is —C₂H₅, including species 17-(3-pyridinyl)-19-nor-19-ethylandrost-5,16-diene-3β,7β-diol, which is

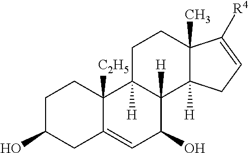

where R⁴ is 3-pyridinyl, 17-(3-pyridinyl)-19-nor-19-ethylandrost-5,16-diene-3α,7β-diol or 17-(3-pyridinyl)-19-nor-19-ethylandrost-5,16-diene-3β,7β-diol-16-acetate.

49A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

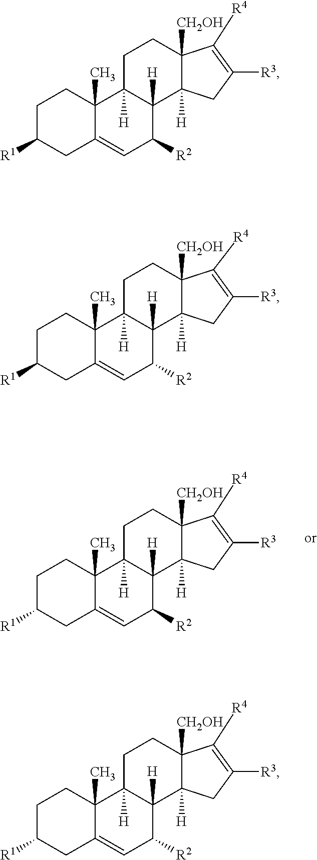

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl, including —CH₃, —C₂H₅ or —CH₂CH₂OH, or optionally wherein the compound is an analog of a compound named in enumerated embodiment 4A, 5A, 6A 7A, 8A, 9A, 10A, 11A, 12A, 13A, 14A, 15A, 16A, 17A, 18A or 19A, wherein in the analog, R⁵ is —CH₂OH, including species 17-(3-pyridinyl)androst-5,16-diene-3β,7β,18-triol, 17-(3-pyridinyl)-7β-methylandrost-5,16-diene-3β,18-diol, 17-(3-pyridinyl)-7β-methylandrost-5,16-diene-3β,18-diol-16-methyl ether or 17-(3-pyridinyl)-7α-methylandrost-5,16-diene-3α,18-diol-16-methyl ether.

50A. The compound of embodiment 44A, 45A, 46A, 47A, 48A or 49A wherein R² is —OH.

51A. The compound of embodiment 44A, 45A, 46A, 47A, 48A or 49A wherein R¹ is —OH or ═O and R² is —OH.

52A. The compound of embodiment 44A, 45A, 46A, 47A, 48A or 49A wherein (a) R³ is —CH₃ and R² is —OH, or (b) R³ is —CF₃, —C₂H₅, —CH₂CH₂OH or —CH₂CH₂CH₃ and R² is —OH.

53A. A formulation comprising one or more excipients and a compound of any of embodiments 23A-52A.

54A. The formulation of embodiment 52A wherein the formulation is for oral administration, wherein the unit dosage form of the formulation is a tablet, capsule, caplet or gelcap.

55A. The formulation of embodiment 52 wherein the formulation is for parenteral administration, including a sterile solution or a sterile suspension.

56A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

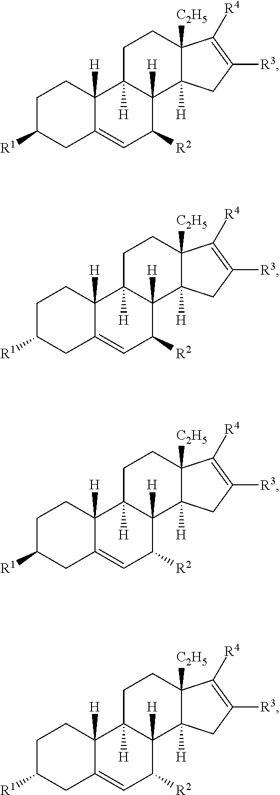

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl, including —CH₃, —C₂H₅ or —CH₂CH₂OH, or optionally wherein the compound is an analog of a compound named in enumerated embodiment 4A, 5A, 6A, 7A, 8A, 9A, 10A, 11A, 12A, 13A, 14A, 15A, 16A, 17A, 18A or 19A, wherein in the analog, R⁵ is —C₂H₅ and R⁶ is —H, including species 17-(3-pyridinyl)-18-nor-18-ethylandrost-5,16-diene-3β,7α-diol, which is

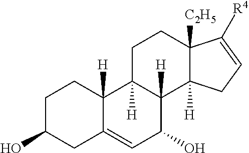

where R⁴ is 3-pyridinyl, 17-(3-pyridinyl)-18,19-dinor-18-ethylandrost-5,16-diene-3α,7α-diol, 17-(3-pyridinyl)-18,19-dinor-18-ethylandrost-5,16-diene-3β,7β-diol-16-acetate, 17-(3-pyridinyl)-18,19-dinor-18-ethyl-7β-ethylandrost-5,16-diene-3β-ol or 17-(3-pyridinyl)-18,19-dinor-18-ethylandrost-5,16-diene-3β,7α-diol-16-methyl ether.

57A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

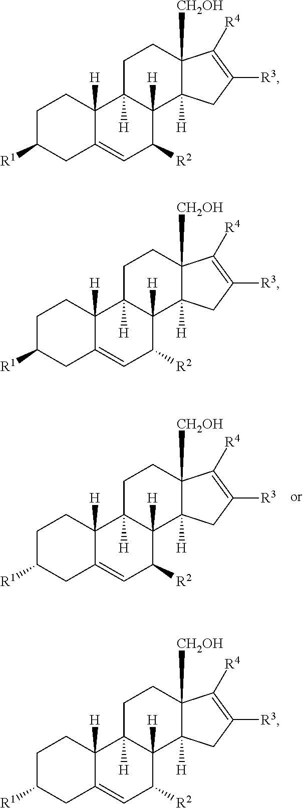

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl including —CH₃, —C₂H₅ or —CH₂CH₂OH, or optionally wherein the compound is an analog of a compound named in enumerated embodiment 4A, 5A, 6A, 7A, 8A, 9A, 10A, 11A, 12A, 13A, 14A, 15A, 16A, 17A, 18A or 19A, wherein in the analog, R⁵ is —CH₂OH and R⁶ is —H, including species 17-(3-pyridinyl)-19-nor-androst-5,16-diene-3β,7β,18-triol, 17-(3-pyridinyl)-19-nor-7β-methylandrost-5,16-diene-3β,18-diol, 17-(3-pyridinyl)-19-nor-7β-methylandrost-5,16-diene-3β,18-diol-16-methyl ether or 17-(3-pyridinyl)-19-nor-7α-methylandrost-5,16-diene-3α,18-diol-16-methyl ether.

58A. The compound of embodiment 23A, 24A, 25A, 26A, 27A, 28A, 29A, 30A, 31A, 32A, 33A, 34A, 35A, 36A, 37A, 38A, 39A, 40A, 41A, 42A or 43A having the structure

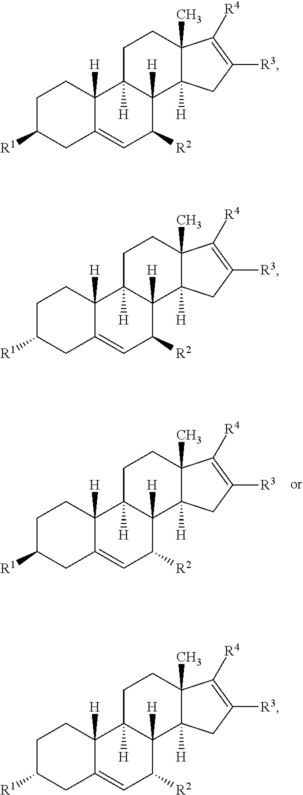

optionally wherein (a) R¹ and R² are —OH and R³ is —H, (b) R¹ is ═O, R² is —OH and R³ is —H, (c) R¹ and R² are —OH and R³ is —CH₃, (d) R¹ is ═O, R² is —OH and R³ is —CH₃, or (e) R¹ is —OH, R² is —H and R³ is C1-4 optionally substituted alkyl, including —CH₃, —C₂H₅ or —CH₂CH₂OH, or optionally wherein the compound is an analog of a compound named in enumerated embodiment 4A, 5A, 6A, 7A, 8A, 9A, 10A, 11A, 12A, 13A, 14A, 15A, 16A, 17A, 18A or 19A, wherein in the analog, R⁶ is —H.

59A. A formulation comprising one or more excipients and a compound of embodiment 56A, 57A or 58A.

60A. The formulation of embodiment 59A wherein the formulation is for oral administration, wherein the unit dosage form of the formulation is a tablet, capsule, caplet or gelcap.

61A. The formulation of embodiment 59A wherein the formulation is for parenteral administration, including a sterile solution or a sterile suspension.

What is claimed is:

1. A method to identify a compound comprising
(a) administering a test compound to a mammal(s) for a sufficient period of time to obtain treated mammal(s);
(b) measuring systemic levels of one or more cholesterol metabolites in the treated mammal(s); and
(c) selecting the compound of step (b) that decreases the systemic levels of one or more cholesterol metabolites in the treated mammal(s), whereby a compound having a potential to treat a cancer, optionally a neuroendocrine cancer is identified, wherein the test compound of step (a) is 17β-ethynyl-5α-androstane-3α-17β-diol or has the structure 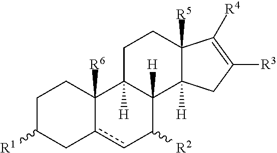
or a salt thereof; wherein the dotted line is a double bond or hydrogen is present at the 5-position in the α-configuration,
R¹ is —OH, —SH, ═O, an optionally substituted ester, wherein the ester is —O—C(O)-optionally substituted C1-7 alkyl or O—C(O)-optionally substituted aryl, optionally acetate, propionate or benzoate, or an optionally substituted ether, wherein the ether is —O-optionally substituted C1-8 alkyl, optionally —OCH₃, —OC₂H₅, —OCH₂CH₂CH₃, —OCH₂CH₂OH, —OCH₂CH₂CH₂OH or —OCH(CH₃)₂;
R² is —OH, —SH, ═O, an optionally substituted ester, wherein the ester is —O—C(O)-optionally substituted C1-7 alkyl or O—C(O)-optionally substituted aryl, optionally acetate, propionate or benzoate, an optionally substituted ether, wherein the ether is —O-optionally substituted C1-8 alkyl, optionally methoxy or ethoxy, or an optionally substituted C1-8 alkyl group, wherein the alkyl group is —CH₃, —CF₃, —C₂H₅, —CH₂CH₂OH, —CH₂CH₂CH₃, —CH₂CH₂CH₂OH or —OCH(CH₃)₂, or R² is —H when (i) R³ is not —H, (ii) R⁵ is —C₂H₅ or —CH₂OH or (iii) R⁶ is —H, —C₂H₅ or —CH₂OH;
R³ is —H, —OH, optionally substituted C1-8 alkyl, optionally methyl, ethyl, n-propyl, i-propyl or 3-hydroxy-n-propyl, halogen, an optionally substituted ester, wherein the ester is —O—C(O)-optionally substituted C1-7 alkyl or —O—C(O)-optionally substituted C1-7 aryl, optionally acetate, propionate or benzoate, an optionally substituted ether, wherein the ether is —O-optionally substituted C1-8 alkyl, optionally —OCH₃, —OC₂H₅, —OCH₂CH₂CH₃, —OCH₂CH₂OH, —OCH₂CH₂CH₂OH or —OCH(CH₃)₂), or an optionally substituted C1-8 alkyl group, wherein the alkyl group is —CH₃, —CF₃, —C₂H₅, —CH₂CH₂OH, —CH₂CH₂CH₃, —CH₂CH₂CH₂OH or —CH(CH₃)₂;
R⁴ is a C-linked ring or an N-linked ring, wherein the ring is a 5- or 6-membered ring;
R⁵ is —CH₃, —C₂H₅ or —CH₂OH; and
R⁶ is —H, —CH₃, —C₂H₅ or —CH₂OH.

2. The method of claim 1 wherein the cholesterol metabolite is one or more of testosterone, dihydrotestosterone, 4-androstenedione, 5-androstenediol, 5α-androstane-3α,17β-diol, 5α-androstane-3β,17β-diol, estradiol, estrone, dehydroepiandrosterone (DHEA), pregnenolone, progesterone and cortisol.

3. The method of claim 1 wherein the cholesterol metabolite is 5α-androstane-3α,17β-diol or 5α-androstane-3β,17β-diol.

4. The method of claim 1 wherein the cholesterol metabolite is dehydroepiandrosterone (DHEA), testosterone, dihydrotestosterone, 4-androstenedione or 5-androstenediol.

5. The method of claim 1 wherein the cholesterol metabolite is pregnenolone, progesterone or cortisol.

6. The method of claim 1 wherein the cholesterol metabolite is 17-hydroxypregnenolone, 11-deoxycortisol or cortisol.

7. The method of claim 1 wherein the compound has the structure 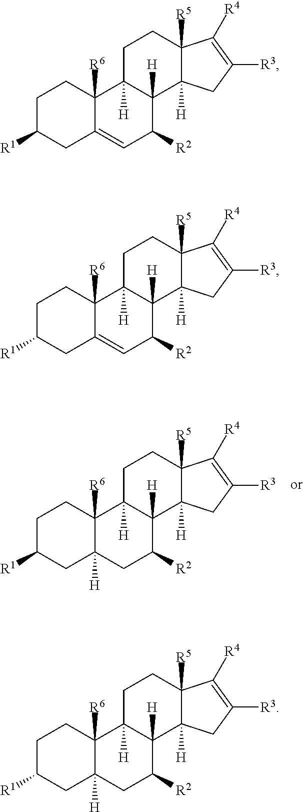
wherein
R¹ is —OH or an ester;
R² is —H, —OH or ═O;
R³ is —OH, halogen, an ester, an ether or an alkyl group; and
R⁴ is a C-linked ring or an N-linked ring, wherein the C-linked ring is a heterocycle.

8. The method of claim 6 wherein R⁴ is 1-furanyl, 2-furanyl, 1-oxolane, 2-oxolane, 1-thiophene, 2-thiophene, 1-pyrrole, 2-pyrrole, 3-pyrrole, 1-pyrrolidine, 2-pyrrolidine, 3-pyrrolidine, 2-thiazolyl, 3-thiazolyl, 4-thiazolyl, 5-thiazolyl, 1-pyranyl, 2-pyranyl or 3-pyranyl.

9. The method of claim 7 wherein R⁴ is —N-pyrrolidine, —N1-pyrazolone, —N2-pyrazolone, —N-imidazolidin-2-one, —N1-imidazole, —N1-4,5-dihydroimidazole, —N-morpholine, —N1-pyridine, —N-piperidine, —N-piperazine, optionally substituted at N4 with optionally substituted alkyl, aryl or heteroaryl, —N-indole, —N-indoline or —N-quinolidine.

10. The method of claim 7 wherein R⁴ is optionally substituted

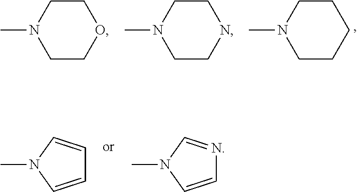

11. The method of claim 6 wherein R⁴ is optionally substituted

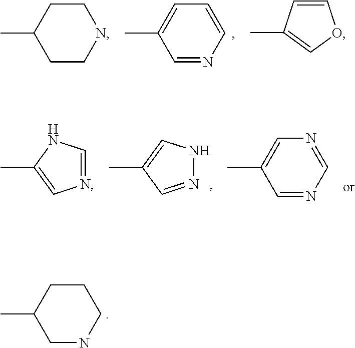

12. The method of claim 7 wherein R⁴ is (1) —N-pyridine or —N-pyrimidinyl, (2) —1-pyridyl, -2-pyridyl, -3-pyridyl, -1-pyrimidinly, -4-pyrimidinly or -5-pyrimidinly, (3) —N-piperidinyl, -1-piperidinyl, -2-piperidinyl, -3-piperidinyl, or (4) —N-imidazole, -2-imidazole or -4-imidazole.

13. The method of claim 1 wherein the compound has the structure

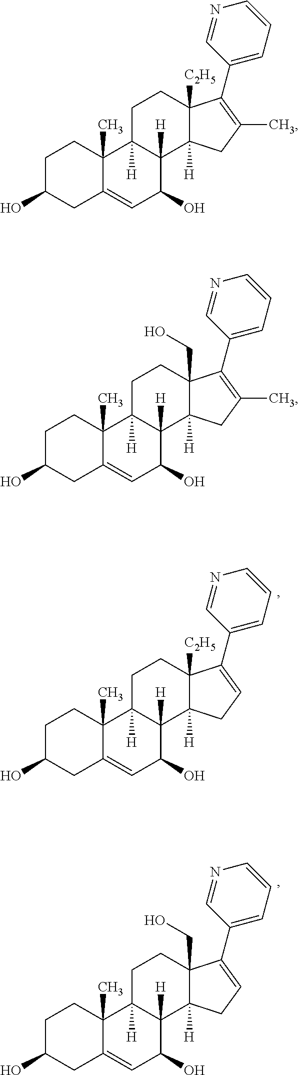

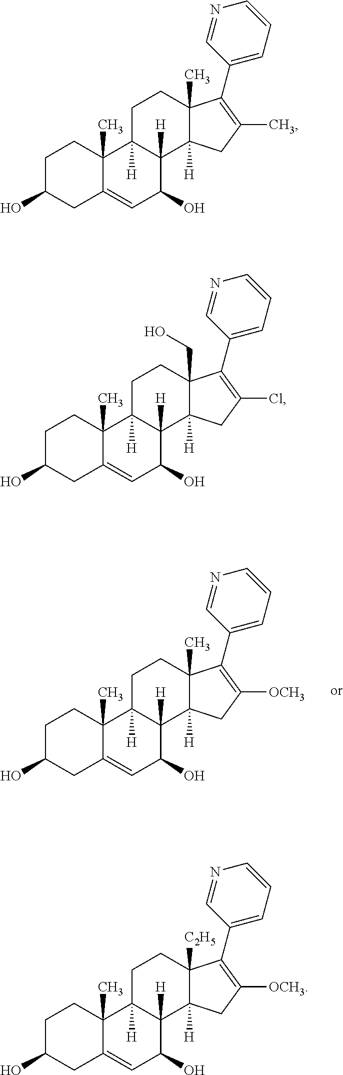

14. The method of claim 1 wherein the compound has the structure

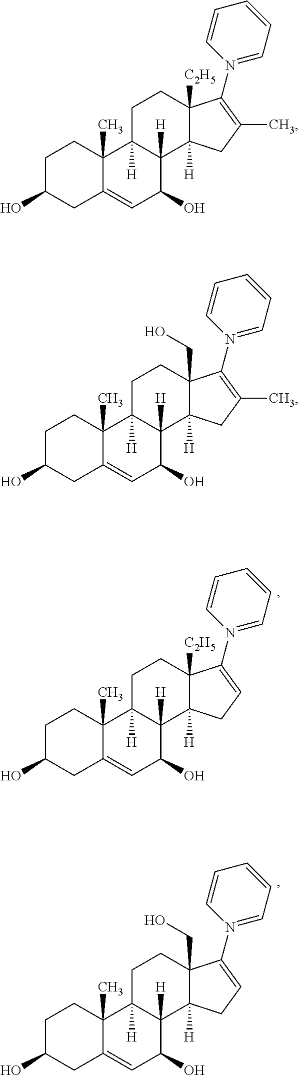

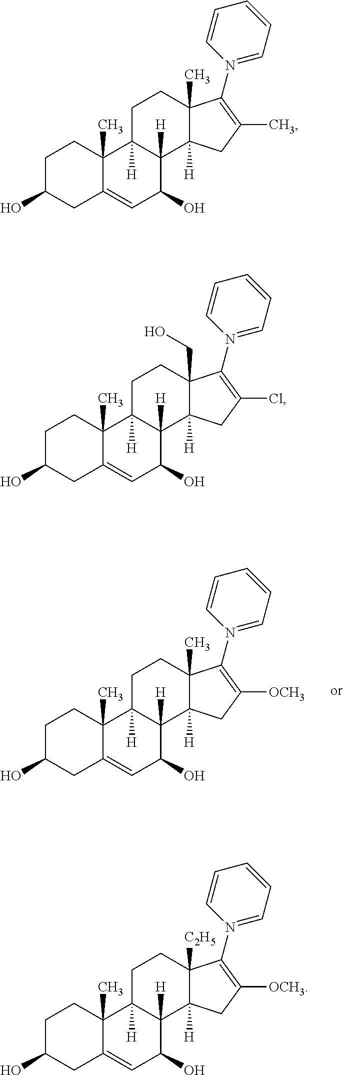

15. The method of claim 1 wherein the compound has the structure

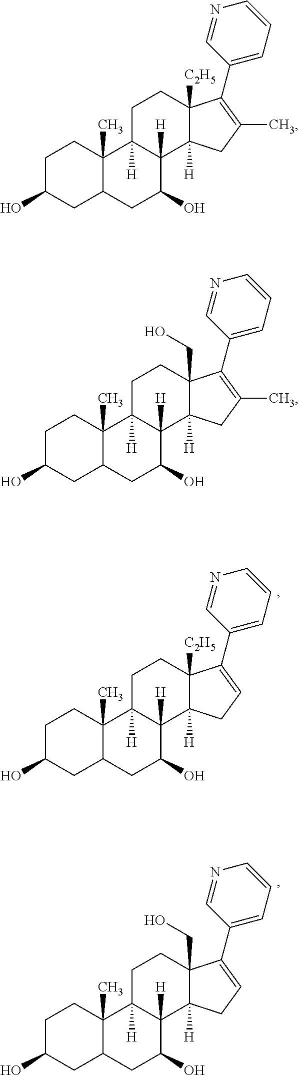

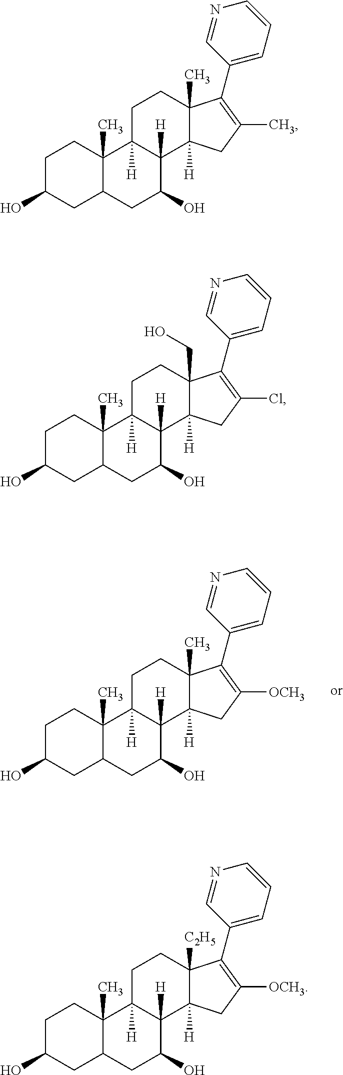

16. The method of claim 1 wherein the compound has the structure

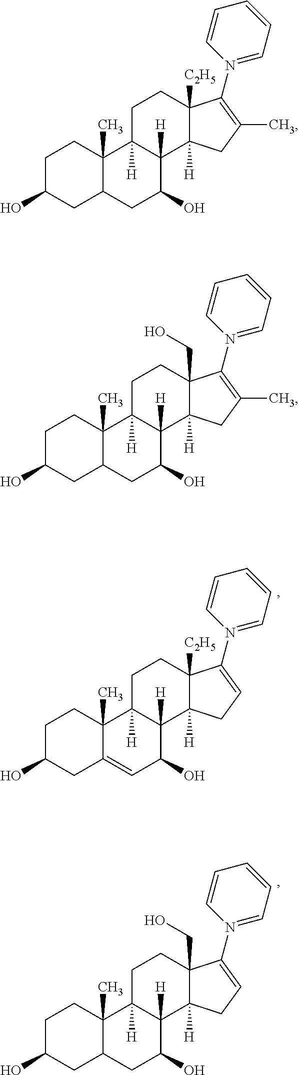

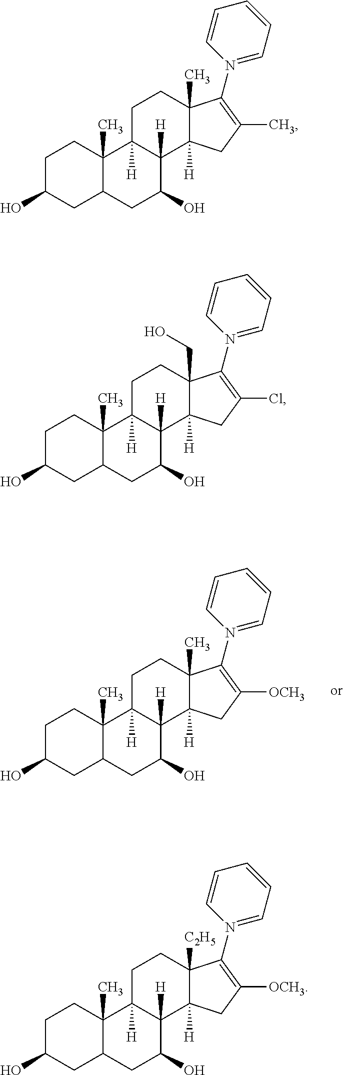

17. The method of claim 1 wherein the compound has the structure

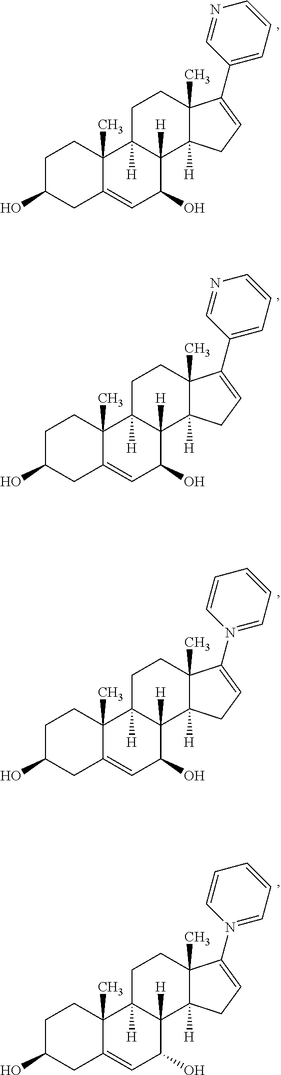

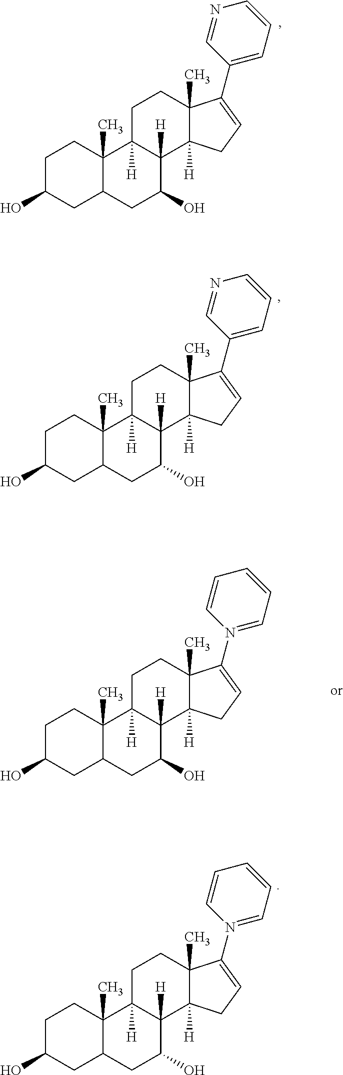

18. The method of claim 13 wherein the hydroxyl at the 7-position is replaced with —OCH₃, optionally wherein the compound is the first, second or third compound shown in claim 13.

19. The method of claim 14 wherein the hydroxyl at the 7-position is replaced with —OCH₃, optionally wherein the compound is the first, second or third compound shown in claim 14.

20. The method of claim 15 wherein the hydroxyl at the 7-position is replaced with —OCH₃, optionally wherein the compound is the first, second or third compound shown in claim 15.

21. The method of claim 16 wherein the hydroxyl at the 7-position is replaced with —OCH₃, optionally wherein the compound is the first, second or third compound shown in claim 16.

22. The method of claim 17 wherein the hydroxyl at the 7-position is replaced with —OCH₃, optionally wherein the compound is the first, second or third compound shown in claim 17.
